(19) United States
(12) Patent Application Publication
(10) Pub. No.: US 2026/0327605 A1
(43) Pub. Date: Oct. 22, 2026

(54) INTEGRATED TRANSCRIPTION AND REVERSE TRANSCRIPTION (ITART)

(71) Applicant: The Broad Institute, Inc., Cambridge, MA (US)

(72) Inventors: Aziz AL'KHAFAJI, Cambridge, MA (US); Alexandre MELNIKOV, Cambridge, MA (US); Allison BROOKHART, Cambridge, MA (US)

(73) Assignee: The Broad Institute, Inc., Cambridge, MA (US)

(21) Appl. No.: 19/651,438

(22) Filed: Apr. 17, 2026

Related U.S. Application Data
(60) Provisional application No. 63/791,348, filed on Apr. 18, 2025.
(60) Provisional application No. 63/853,242, filed on Jul. 29, 2025.

(51) Int. Cl. C12N 15/10

(52) U.S. Cl. CPC C12N 15/1065; C12N 15/1096

(57) ABSTRACT

This disclosure provides a method for amplifying a DNA molecule that addresses the limitations of traditional in vitro transcription (IVT) followed by reverse transcription (RT). The method of the disclosure combines IVT and RT into a single, highly efficient one-step linear amplification process and is called Integrated Transcription and Reverse Transcription (ITART). This innovative approach rapidly converts newly synthesized IVT RNA into more stable cDNA, significantly improving RNA yield and integrity. This method offers a robust solution for amplifying low-quality or low-abundance DNA samples, making it a valuable tool for various applications in molecular biology and genetics.

### CROSS-REFERENCE TO RELATED APPLICATIONS

This application claims the benefit under 35 U.S.C. § 119(e) of U.S. Provisional Application No. 63/791,348, filed Apr. 18, 2025, and U.S. Provisional Application No. 63/853,242, filed Jul. 29, 2025, the entire contents of which are incorporated herein by reference.

### BACKGROUND

In vitro transcription (IVT), followed by first-strand cDNA synthesis through reverse transcription (RT), is a two-step process for generating cDNA from a DNA template with a predefined promoter upstream of the sequences of interest. This process is widely utilized for sequencing, gene expression studies, and cloning. However, RNA degradation, particularly during the extended IVT times required to amplify small amounts of template, affects RNA yield and integrity, and leads to high background and low reproducibility in cDNA generation. Therefore, improved methods are needed, especially for low-abundance or low-quality samples.

### SUMMARY

The details of one or more embodiments of the present disclosure are set forth in the description below. Additional features or advantages of the present disclosure will be apparent from the following drawings and detailed description of several embodiments, as well as the appended claims.

An aspect of the disclosure is directed to an isothermal method for amplifying a DNA molecule in a sample, comprising producing an RNA copy of the DNA molecule using an RNA polymerase, and producing a complementary DNA (cDNA) copy of the RNA copy using a reverse transcriptase, thereby linearly amplifying the DNA molecule, wherein producing an RNA copy and producing a cDNA copy are carried out as a one pot reaction.

In some embodiments, the RNA polymerase and the reverse transcriptase are active at the same time, and producing an RNA copy and producing a cDNA copy occur simultaneously.

In some embodiments, reverse transcribing the RNA copy into the cDNA copy is done while the RNA copy is being produced.

In some embodiments, the reverse transcriptase creates a DNA copy of RNA produced by the RNA polymerase substantially simultaneously with production of the RNA.

In some embodiments, the DNA molecule comprises an RNA polymerase promoter and a reverse transcriptase primer binding region.

In some embodiments, the RNA polymerase promoter and the reverse transcriptase primer binding region are added to the DNA molecule using a transposase.

In some embodiments, the transposase is selected from a Tn5 transposase, a Tn3 transposase, a Tn7 transposase, a Tn10 transposase, a PiggyBac transposase, a Sleeping Beauty transposase, or a Tol2 transposase.

In some embodiments, the RNA polymerase promoter is introduced by an adapter, a primer, or a transposase.

In some embodiments, the RNA polymerase promoter is a unidirectional promoter or a bidirectional promoter.

In some embodiments, the RNA polymerase promoter is a T7 promoter.

In some embodiments, the one pot reaction is carried out in a reaction buffer that comprises a ribonucleotide triphosphate (NTP) to deoxyribonucleotide triphosphate (dNTP) ratio (NTP: dNTP) of between 5:1 and 1:2.

In some embodiments, the NTP: dNTP ratio is 1:1.

In some embodiments, the reaction buffer comprises between 2-5 mM Mg²⁺ or 0.5-10 mM Mg²⁺.

In some embodiments, the DNA molecule further comprises at least one molecular barcode.

In some embodiments, the molecular barcode identifies a partition or a cell of origin.

In some embodiments, the RNA polymerase is selected from a T7 RNA polymerase, a T3 RNA polymerase, or an SP6 RNA polymerase.

In some embodiments, the reverse transcriptase lacks RNase activity and/or exonuclease activity.

In some embodiments, the reverse transcriptase is selected from a Maxima™ H-Minus reverse transcriptase, an HIV reverse transcriptase, avian myeloblastosis virus (AMV) reverse transcriptase, a Moloney murine leukemia virus (M-MuLV or MMLV) based reverse transcriptase, or a Group II Intron transcriptase.

In some embodiments, the method further comprises contacting abortive RNA transcripts with one or more blocking oligonucleotides that reduce reverse transcription initiated from the abortive RNA transcripts.

In some embodiments, the one or more blocking oligonucleotides hybridize to abortive RNA transcripts produced during in vitro transcription.

In some embodiments, the DNA molecule comprises genomic DNA or cDNA.

In some embodiments, the DNA molecule is obtained from a single cell or a small population of cells.

In some embodiments, the sample comprises a biological sample or an environmental sample.

In some embodiments, the amplifying is achieved at a constant temperature between about 20° C. and about 65° C.

In some embodiments, the amplifying is achieved at about 37° C.

In some embodiments, the linearly amplified DNA molecule is used directly in a sequencing workflow.

In some embodiments, the linearly amplified DNA molecule is used in the sequencing workflow without a PCR amplification step.

In some embodiments, the linearly amplified DNA molecule is converted to double-stranded DNA for use in a sequencing workflow.

In some embodiments, the method further comprises amplifying the linearly amplified DNA molecule using a polymerase chain reaction (PCR) using less than 20 cycles.

In some embodiments, the cDNA copy comprises the RNA polymerase promoter sequence.

In some embodiments, the cDNA copy is used as a template for producing a second RNA copy using the RNA polymerase, and producing a second complementary DNA copy of the second RNA copy using the reverse transcriptase, wherein producing the second RNA copy and producing the second complementary DNA copy are carried out in the same one pot reaction.

In some embodiments, the second RNA copy is produced from the RNA polymerase promoter sequence present in the cDNA copy.

In some embodiments, the RNA polymerase recognizes the RNA polymerase promoter sequence in the cDNA copy and initiates transcription to produce the second RNA copy.

In some embodiments, the RNA polymerase promoter sequence is transcribed as part of the RNA copy and is present in the cDNA copy following reverse transcription.

In some embodiments, the cDNA copy undergoes multiple rounds of transcription and reverse transcription within the same one pot reaction, thereby achieving iterative linear amplification.

An aspect of the disclosure is directed to an isothermal method for preparing a sequencing library from a sample said method comprising: providing a nucleic acid molecule, said nucleic acid molecule comprising: a feature sequence and an RNA polymerase promoter upstream of the feature sequence; producing an RNA transcript of the feature sequence using an RNA polymerase; and producing a complementary DNA (cDNA) copy of the RNA transcript using a reverse transcriptase, wherein producing the RNA transcript and producing the cDNA copy are carried out as a one pot reaction, thereby amplifying and capturing the feature sequence.

In some embodiments, the sample comprises a single-cell sequencing library.

In some embodiments, the sample comprises a bulk sequencing library.

In some embodiments, the sample is a partitioned sample.

In some embodiments, the nucleic acid molecule is provided in a partition.

In some embodiments, the partition is a droplet, an emulsion, an encapsulation, a microwell, or other compartment that preserves an association between the feature sequence and a cell or partition of origin.

In some embodiments, the RNA polymerase and the reverse transcriptase are active at the same time, and producing the RNA transcript and producing the cDNA copy occur substantially simultaneously.

In some embodiments, producing the RNA transcript and producing the cDNA copy are carried out at a constant temperature.

In some embodiments, the feature sequence is genomically encoded.

In some embodiments, the feature sequence is not natively transcribed prior to producing the RNA transcript.

In some embodiments, the RNA polymerase promoter is a T7 promoter.

In some embodiments, the feature sequence is selected from a guide RNA sequence, a DNA barcode sequence, an open reading frame sequence, an overexpression construct sequence, or a sequence of interest.

In some embodiments, the method further comprises hybridizing a feature-specific primer to the RNA transcript or the cDNA copy to enrich a region of interest downstream of the RNA polymerase promoter.

In some embodiments, the sequencing library further comprises native RNA, and the method captures the feature sequence together with the native RNA.

In some embodiments, the partition comprises a barcode such that the amplified and captured feature sequence remains associated with a cell or partition of origin.

In some embodiments, the feature sequence has a uniform size or a narrow size distribution.

In some embodiments, the method is used in a single-cell CRISPR screening workflow.

In some embodiments, the single-cell CRISPR screening workflow is a Perturb-seq workflow.

In some embodiments, the feature sequence comprises a guide RNA sequence.

In some embodiments, the method is used in a bulk CRISPR screening workflow.

In some embodiments, the bulk CRISPR screening workflow involves sequencing of multiple samples in a single reaction vessel.

In some embodiments, the cDNA copy comprises the RNA polymerase promoter sequence.

In some embodiments, the cDNA copy is used as a template for producing a second RNA transcript using the RNA polymerase, and producing a second complementary DNA copy of the second RNA transcript using the reverse transcriptase, wherein producing the second RNA transcript and producing the second complementary DNA copy are carried out in the same one pot reaction.

In some embodiments, the second RNA transcript is produced from the RNA polymerase promoter sequence present in the cDNA copy.

In some embodiments, the feature sequence undergoes iterative linear amplification through multiple rounds of transcription and reverse transcription.

In some embodiments, the RNA polymerase promoter sequence is transcribed as part of the RNA transcript and is present in the cDNA copy following reverse transcription.

In some embodiments, the feature sequence is a guide RNA sequence used in a single-cell CRISPR screening workflow.

In some embodiments, the guide RNA sequence comprises the RNA polymerase promoter sequence, and the cDNA copy of the guide RNA sequence serves as a template for subsequent rounds of transcription and reverse transcription.

An aspect of the disclosure is directed to a method for capturing a feature sequence in an RNA-based library preparation workflow, said method comprising: providing a DNA molecule comprising an RNA polymerase promoter upstream of a feature sequence; producing an RNA transcript of the feature sequence using an RNA polymerase; and producing a complementary DNA (cDNA) copy of the RNA transcript using a reverse transcriptase, wherein producing the RNA transcript and producing the cDNA copy are carried out as a one pot isothermal reaction, thereby rendering the feature sequence captureable in the RNA-based library preparation workflow.

In some embodiments, the feature sequence is not natively transcribed.

In some embodiments, the feature sequence is selected from a guide RNA sequence, a DNA barcode sequence, an open reading frame sequence, an overexpression construct sequence, or a sequence of interest.

In some embodiments, a feature-specific primer is used to enrich a region of interest downstream of the RNA polymerase promoter.

In some embodiments, the RNA polymerase promoter is a T7 promoter.

In some embodiments, the RNA transcript and the cDNA copy are generated in a partition.

In some embodiments, the partition is associated with a single cell.

In some embodiments, the RNA transcript and the cDNA copy are generated in a bulk reaction.

In some embodiments, the cDNA copy comprises the RNA polymerase promoter sequence.

In some embodiments, the cDNA copy is used as a template for producing a second RNA transcript using the RNA polymerase, and producing a second complementary DNA copy of the second RNA transcript using the reverse transcriptase, wherein producing the second RNA transcript and producing the second complementary DNA copy are carried out in the same one pot isothermal reaction.

In some embodiments, the feature sequence undergoes iterative linear amplification through multiple rounds of transcription and reverse transcription.

In some embodiments, the RNA polymerase promoter sequence is encoded in the RNA transcript and is present in the cDNA copy following reverse transcription.

An aspect of the disclosure is directed to a composition comprising an RNA polymerase and a reverse transcriptase in the same reaction buffer, wherein the RNA polymerase and the reverse transcriptase are active in the same reaction buffer at the same time.

In some embodiments, the RNA polymerase is selected from a T7 RNA polymerase, a T3 RNA polymerase, or an SP6 RNA polymerase.

In some embodiments, the reverse transcriptase is selected from the group consisting of: a Maxima™ H-Minus reverse transcriptase, an HIV reverse transcriptase, avian myeloblastosis virus (AMV) reverse transcriptase, a Moloney murine leukemia virus (M-MuLV or MMLV) based reverse transcriptase, or a Group II Intron reverse transcriptase.

In some embodiments, the composition further comprises one or more primers configured to hybridize to a feature sequence transcribed from a DNA molecule, said DNA molecule comprising an RNA polymerase promoter upstream of the feature sequence.

In some embodiments, the composition further comprises one or more blocking oligonucleotides configured to hybridize to abortive RNA transcripts.

An aspect of the disclosure is directed to a kit comprising: a composition and instructions for use, said composition comprising an RNA polymerase and a reverse transcriptase in the same reaction buffer, wherein the RNA polymerase and the reverse transcriptase are active in the same reaction buffer at the same time.

In some embodiments, the instructions are for use in a partition-based single-cell sequencing workflow.

In some embodiments, the instructions are for use in a bulk sequencing workflow.

In some embodiments, the instructions are for capturing a guide RNA sequence, a DNA barcode sequence, an open reading frame sequence, an overexpression construct sequence, or a sequence of interest.

### BRIEF DESCRIPTION OF THE DRAWINGS

The present disclosure is illustrated by way of example, and not by way of limitation, in the figures of the accompanying drawings and in which like reference numerals refer to similar elements and in which:

FIGS. 1A-1B compare standard IVT and RT to the novel ITART method described herein. FIG. 1C shows a schematic workflow of IVT followed by RT and PCR from a low input template. IVT: in vitro transcription, SPRI: Solid phase reversible immobilization, FSS: first strand synthesis in reverse transcription, PCR: Polymerase Chain Reaction. FIG. 1D shows a graph of direct PCR from sorted nuclei (about 4 kb). FIG. 1E shows a graph of IVT RNA from 125 nuclei after 48 hours of IVT (<2 kb), which illustrates that most IVT RNA is degraded after an extended reaction time. FIG. 1F shows a graph illustrating that after RT, the cDNA (about 4 kb) is further amplified by PCR. PCR products of various sizes are produced, indicating that the original RNA has been degraded. This significantly reduces the quality of the final product.

FIG. 2A demonstrates the efficiencies of T7 RNA polymerase and RTase in the reaction buffer with NTP and dNTP. FIG. 2B shows qPCR of ITART cDNA from 1 kb template. FIG. 2C shows size distribution of ITART cDNA after PCR amplification. FIGS. 2D-2F show a high concentration of Mg²⁺ increases RNA fragmentation during IVT. FIG. 2D is a graph showing IVT-produced RNA was incubated in a buffer containing 4 mM Mg²⁺ for 0, 2, 6, 24, 48, or 72 hours at 37° C. With increasing time, the amount of intact RNA in the buffer decreased. FIG. 2E is a graph showing the rate of RNA Fragmentation in 4 mM Mg²⁺. FIG. 2F is a graph showing intact RNA concentration after 24 hours incubation at 37° C. with different concentrations of Mg²⁺.

FIG. 3A shows an embodiment of ITART. During ITART, a DNA template is linearly amplified by simultaneous in vitro transcription and reverse transcription in the same reaction. FIG. 3B shows another embodiment of ITART. T7: T7 reverse transcriptase primer. AMP: Amplification primer. BC: Barcode. PIPE Tag Sequencing: Paired index position-encoded sequencing. FIG. 3C shows a non-limiting example of lambda template DNA for ITART.

FIG. 4A is a bar chart showing a first strand synthesis (FSS) during RT is less sensitive to the presence of ribonucleotide triphosphates (NTPs) than IVT is to deoxyribonucleotide triphosphates (dNTPs). Higher amounts of dNTP inhibited the RNA polymerase action. FIG. 4B shows the raw data of the IVT experiment with different NTP:dNTP ratios. FIG. 4C shows the raw data of the Maxima FSS experiment with different NTP:dNTP ratios. A great variance was observed in sample size during FSS, indicating degradation of the original template.

FIG. 5A and FIG. 5B show that in the absence of RT primers, abortive transcripts can initiate off-target reverse transcription. FIGS. 5C-5D show a schematic and data illustrating that blocking oligos can prevent off-target reverse transcription from abortive transcripts.

FIGS. 6A-6E show ITART can faithfully amplify extremely low amounts of DNA. FIG. 6A shows 5 ng, 500 pg, or 50 pg of template were used in a 2 hour ITART reaction, and the resulting DNA amounts were measured by fluorescence. 2 hours of ITART was able to produce a detectable product of the correct size from as low as 500 pg of template. FIG. 6B shows products from FIG. 6A were diluted 1:50 and used in a qPCR reaction to determine the amount of product. The assay was able to detect a product from a 500 pg starting template after the product dilution 1:50 in less than 20 PCR cycles. FIG. 6C shows quantification of ITART cDNA Amplicons. FIG. 6D shows IVT yield from 2 hr incubation with 10 ng lambda template. FIG. 6E shows lowering NTP concentration and changing to a more compatible buffer results in a ~2× decrease in IVT yield. Hi-temp T7 is 10× less efficient than std T7 in the same buffer.

FIGS. 7A-7C show an ITART reaction in partition format with feature capture. FIG. 7A shows a cell lysed and primers immediately bind to target nucleic acids and/or genomic DNA that is exposed. FIG. 7B shows that RNA polymerase initiates transcription from feature loci. Target specific primers bind to the newly-transcribed feature. FIG. 7C shows that reverse transcription generates cDNA of native mRNAs and target captured features.

### DETAILED DESCRIPTION

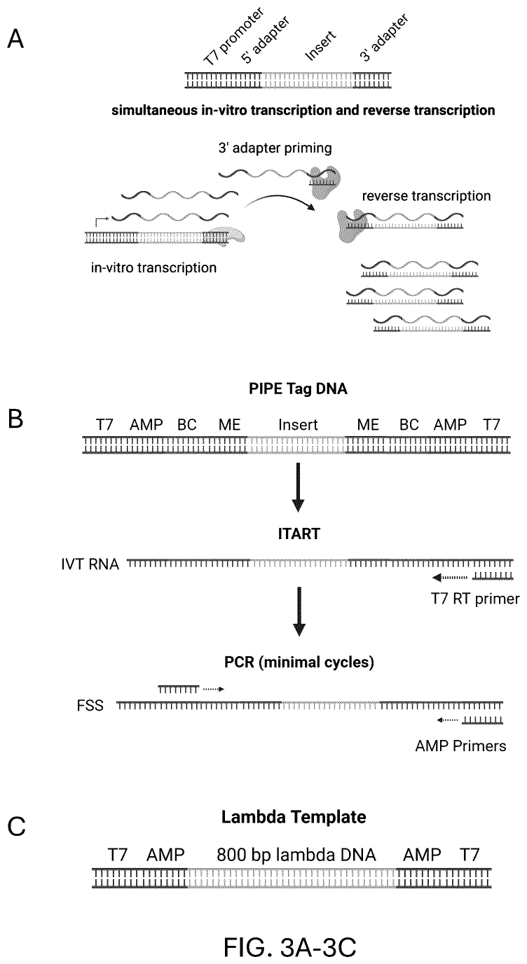

The present disclosure describes a method called “Integrated Transcription and Reverse Transcription (ITART),” which overcomes the disadvantages of IVT followed by RT by combining IVT and RT in a highly efficient one-step linear amplification. In this reaction, RT rapidly converts the de novo synthesized IVT RNA into more stable cDNA (FIG. 3A). The ITART method is suitable for DNA amplification workflow steps in genomic and transcriptomic assays such as single-cell genome sequencing, single-cell transcriptomics, RNA-seq, transposon-based techniques, and multiplex protein analysis via proximity extension assays.

The disclosure addresses a technical problem in nucleic acid amplification arising from the use of sequential in vitro transcription (IVT) followed by reverse transcription (RT) in separate reaction steps. In conventional methods, IVT is performed first to generate RNA amplicons from a DNA template containing an RNA polymerase promoter. The RNA product is then purified or otherwise handled before being subjected to a separate RT reaction to generate complementary DNA. During the IVT step, particularly when extended reaction times are required for low-abundance templates, the RNA product is susceptible to degradation through hydrolysis, nuclease activity, and metal-catalyzed fragmentation. High magnesium concentrations, which are often present in IVT reaction buffers to support polymerase activity, can accelerate RNA degradation. As a result, by the time the RT step is performed, a substantial portion of the RNA may have been degraded or fragmented, leading to reduced yield, loss of longer amplicons, and increased background signal from non-specific products.

This problem is particularly pronounced in applications requiring amplification from very small amounts of starting material, such as single-cell genomics, low-input RNA sequencing, and targeted feature capture in partitioned single-cell workflows. In such contexts, the accumulation of degraded RNA during a prolonged IVT reaction can compromise the fidelity and uniformity of the final amplified product. Furthermore, conventional PCR-based amplification methods, while robust, introduce exponential amplification bias, replication errors, and chimeric artifact formation, particularly in workflows involving complex libraries with barcodes and adapter sequences. These biases and artifacts reduce the accuracy of downstream analyses, including genotyping, transcriptome profiling, and perturbation screening.

The disclosure solves this problem by integrating the IVT and RT reactions into a single reaction environment, such that RNA produced by the RNA polymerase is converted into complementary DNA by the reverse transcriptase in real time or near-real time. By carrying out both enzymatic steps simultaneously in the same reaction buffer, the RNA intermediate is stabilized through immediate conversion to DNA, thereby minimizing the opportunity for RNA degradation. The reaction is performed isothermally, without the need for thermal cycling, and employs a shared buffer composition that supports the activity of both the RNA polymerase and the reverse transcriptase. The reaction buffer may contain both ribonucleotide triphosphates and deoxyribonucleotide triphosphates in a balanced ratio, such as between 5:1 and 1:2, to enable concurrent transcription and reverse transcription. The magnesium concentration may be optimized to support enzyme activity while reducing RNA fragmentation, for example in a range between 0.5 mM and 10 mM.

The result is a linear amplification process that preserves the integrity of longer amplicons, reduces background noise, and provides more uniform representation of the starting template compared to conventional sequential IVT and RT or exponential PCR-based methods. The disclosure is particularly advantageous for low-input applications, where template amounts may be as low as picogram levels, and for workflows requiring accurate quantitation of specific features, such as guide RNA capture in Perturb-seq or other single-cell CRISPR screening assays. By eliminating intermediate purification and handling steps, the method also simplifies the workflow and reduces the risk of sample loss or contamination.

In a further aspect, the disclosure addresses a related problem in partitioned single-cell workflows, where accurate capture and quantitation of genomically encoded features is required. In such workflows, cells are compartmentalized into partitions, such as droplets or microwells, and each partition is associated with a unique barcode that identifies the originating cell or partition. A particular challenge arises when the feature of interest, such as a guide RNA sequence in a Perturb-seq experiment, is present in low abundance or is susceptible to cross-contamination from lysed cells in the same sample. Conventional PCR-based amplification of such features can introduce substantial bias, leading to inaccurate assignment of features to cells and reduced reliability of phenotype-genotype associations.

The disclosure solves this problem by applying the integrated transcription and reverse transcription reaction within each partition, such that the feature sequence is amplified and captured post-compartmentalization. A DNA molecule encoding the feature sequence is provided with an RNA polymerase promoter positioned upstream of the feature. During the reaction, the RNA polymerase generates an RNA transcript from the DNA-encoded feature within the partition, and the reverse transcriptase converts the RNA transcript into cDNA in the same reaction. Because the signal-generating transcript is produced within the partition rather than being captured from ambient or pre-existing RNA, cross-cell contamination is reduced. The linear amplification provided by the integrated reaction also reduces amplification bias compared to PCR, resulting in more accurate quantitation and representation of the feature across multiple partitions. The method is compatible with existing barcoding schemes, allowing the captured feature to remain linked to the originating cell or partition throughout downstream sequencing and analysis.

A further advantage of the disclosure is its applicability to features that are not natively transcribed in the cell. By placing an RNA polymerase promoter upstream of a genomically encoded feature, such as a DNA barcode, an overexpression construct, or another engineered sequence element, the feature can be rendered captureable within an RNA-based library preparation workflow. The in vitro transcription generates an RNA intermediate that can be processed using the same reagents and workflows employed for native RNA capture, thereby enabling unified library preparation for both transcriptome profiling and targeted feature capture. This approach is particularly useful when the captured feature has a uniform size or narrow size distribution, as length-dependent biases that may arise in downstream processing steps are minimized.

### DEFINITIONS

Unless otherwise defined herein, scientific and technical terms used in connection with the present disclosure shall have the meanings that are commonly understood by those of ordinary skill in the art (e.g., the skilled artisan). The meaning and scope of the terms are clear; however, in the event of any latent ambiguity, definitions provided herein take precedence over any dictionary or extrinsic definition. Further, unless otherwise required by context, singular terms shall include pluralities, and plural terms shall include the singular. In this disclosure, the use of “or” means “and/or” unless stated otherwise. Furthermore, the use of the term “including,” as well as other forms, such as “includes” and “included,” is not limiting. Also, terms such as “element” or “component” encompass both elements and components comprising one unit and elements and components that comprise more than one subunit unless specifically stated otherwise.

General terminology in cell and molecular biology can be found in “The Merck Manual of Diagnosis and Therapy”, 19th Edition, published by Merck Research Laboratories, 2006 (ISBN 0-911910-19-0); Robert S. Porter et al. (Eds.), The Encyclopedia of Molecular Biology, published by Blackwell Science Ltd., 1994 (ISBN 0-632-02182-9). General terminology definitions in molecular biology are also given in Benjamin Lewin, Genes X, published by Jones & Bartlett Publishing, 2009 (ISBN-10: 0763766321); Kendrew et al. (Eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds., each of which are incorporated herein in their entireties.

Generally, nomenclatures used in connection with, and techniques of, cell and tissue culture, molecular biology, immunology, microbiology, genetics, and protein and nucleic acid chemistry and hybridization described herein are those well-known and commonly used in the art. The methods and techniques of the present disclosure are generally performed according to conventional methods well known in the art and as described in various general and more specific references that are cited and discussed throughout the present disclosure, unless otherwise indicated. Enzymatic reactions and purification techniques are performed according to the manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, delivery, and treatment of subjects.

For the present disclosure to be more readily understood, select terms are defined below.

The singular forms “a”, “an”, and “the” include the plural unless the text clearly indicates otherwise. Similarly, the term “or” is intended to include “and” unless the text clearly indicates otherwise. Although methods and materials similar or equivalent to those described herein can be used in the practice or testing of the present disclosure, suitable methods and materials are described below. The abbreviation “e.g.,” is derived from the Latin phrase “exempli gratia” and is used herein to give a non-limiting example. For this reason, the abbreviation “for example (e.g.)” is synonymous with the term “for example”.

The terms “approximately” or “about,” as may be used interchangeably herein, and as applied to one or more values of interest, refer to a value that is similar to a stated reference value. In certain embodiments, the term “approximately” or “about” refers to a range of values that fall within 15%, 14%, 13%, 12%, 11%, 10%, 9%, 8%, 7%, 6%, 5%, 4%, 3%, 2%, 1%, or less in either direction of (i.e., percentage greater than or percentage less than) the stated reference value unless otherwise stated or otherwise evident from the context (for example, when such number would exceed 100% of a possible value).

As used herein, the term “sample” refers to any composition of matter that contains, or is suspected of containing, nucleic acids, e.g., DNA.

The term “biological sample” as used herein generally refers to a tissue or body fluid (biological fluid) sample derived from a subject. Biological samples can be obtained directly from a subject. The biological sample can be or can comprise one or more nucleic acid molecules, such as deoxyribonucleic acid (DNA) or ribonucleic acid (RNA) molecules. The biological sample may be derived from any organ, tissue, or biological fluid. The biological sample may comprise, for example, body fluids or solid tissue samples. An example of a solid tissue sample is a tumor sample, e.g., a solid tumor biopsy. Body fluids include, for example, blood, serum, plasma, tumor cells, saliva, urine, lymphatic fluid, synovial fluid, interstitial fluid, cerebrospinal fluid, prostate fluid, semen, sputum, mucus, gastric acid, bile, feces, tears, vaginal lavage and derivatives thereof.

A biological sample may be of prokaryotic origin or eukaryotic origin (e.g., insects, protozoa, birds, fish, reptiles). In some embodiments, the biological sample is mammalian (e.g., rat, mouse, cow, dog, donkey, guinea pig, or rabbit). In certain embodiments, the biological sample is of primate origin (e.g., example, chimpanzee, or human).

As used herein, an “environmental sample” refers to any sample that is obtained from the environment. An environmental sample may include liquid samples from a river, lake, pond, ocean, glaciers, icebergs, rain, snow, sewage, reservoirs, tap water, drinking water, etc.; solid samples from soil, compost, sand, rocks, concrete, wood, brick, sewage, etc.; and gaseous samples from the air, underwater heat vents, industrial exhaust, vehicular exhaust, etc. Typically, samples that are not in liquid form are converted to liquid form before analyzing the sample with the present methods.

The term “substantially,” as may be used herein, when used to describe the degree or abundance of an activity, generally refers to the value of the activity as being an amount which is achievable without undue effort. As can be appreciated, this amount may vary depending on the activity being performed, with simpler activities requiring a higher threshold and more complex activities requiring a lower threshold.

As used herein, the term “simultaneously” means at the same time. For instance, when an RNA polymerase and reverse transcriptase catalyze their reactions at the same time, they work simultaneously.

As used herein, the term “substantially simultaneously” means within some predefined time lag (e.g., in the range of seconds) between one action and the other. For instance, when after an RNA polymerase produces an RNA copy of the DNA template, a reverse transcriptase begins to convert the RNA copy into a cDNA copy, both enzymes work substantially simultaneously.

The expression “amplification” or “amplifying” refers to a process by which extra or multiple copies of a particular polynucleotide are formed. Amplification includes methods such as PCR, ligation amplification (or ligase chain reaction, LCR) and other amplification methods. These methods are known and widely practiced in the art. See, e.g., U.S. Pat. Nos. 4,683,195 and 4,683,202 and Innis et al., “PCR protocols: a guide to method and applications” Academic Press, Incorporated (1990) (for PCR); and Wu et al. (1989) Genomics 4:560-569 (for LCR). In general, the PCR procedure describes a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified.

Reagents and hardware for conducting the amplification reaction are commercially available. Primers useful to amplify sequences from a particular gene region are preferably complementary to, and hybridize specifically to sequences in the target region or in its flanking regions and can be prepared using the polynucleotide sequences provided herein. Nucleic acid sequences generated by amplification can be sequenced directly.

The term “amplification reagents” refers to those reagents (deoxyribonucleotide triphosphates, buffer, etc.), needed for amplification except for primers, nucleic acid template, and the amplification enzyme. Typically, amplification reagents along with other reaction components are placed and contained in a reaction vessel (test tube, microwell, etc.). Amplification methods include PCR methods known to those of skill in the art and also include rolling circle amplification (Blanco et al., J. Biol. Chem., 264, 8935-8940, 1989), hyperbranched rolling circle amplification (Lizard et al., Nat. Genetics, 19, 225-232, 1998), and loop-mediated isothermal amplification (Notomi et al., Nuc. Acids Res., 28, e63, 2000) each of which are hereby incorporated by reference in their entireties.

As used herein, the term “linear amplification” refers to an amplification process in which the number of copies of a target nucleic acid sequence increases in direct proportion to the number of reaction cycles or reaction time, rather than exponentially as in polymerase chain reaction (PCR). In linear amplification, each template molecule serves as a substrate for generating product molecules, but the product molecules themselves do not serve as templates for further amplification within the same reaction. This contrasts with exponential amplification methods, where newly synthesized products become templates in subsequent cycles, leading to exponential growth in product quantity. Linear amplification may produce single-stranded complementary DNA (cDNA) copies directly from an RNA template through reverse transcription. The single-stranded cDNA product may be used directly in downstream applications, such as sequencing workflows that accommodate single-stranded nucleic acids, or may be converted to double-stranded DNA (dsDNA) through second-strand synthesis or other enzymatic processes. In some embodiments, the linearly amplified product, whether single-stranded or double-stranded, may be subjected to a limited number of PCR cycles, such as fewer than 20 cycles, fewer than 15 cycles, fewer than 10 cycles, or fewer than 5 cycles, to generate additional copies for downstream analysis. The use of limited-cycle PCR following linear amplification can provide increased product yield while minimizing the amplification bias and error accumulation associated with high-cycle PCR. The term “linear amplification” encompasses both in vitro transcription-based amplification, in which an RNA polymerase generates multiple RNA copies from a DNA template, and reverse transcription-based amplification, in which a reverse transcriptase generates cDNA copies from an RNA template. In the context of the present disclosure, linear amplification through the integrated transcription and reverse transcription (ITART) method involves both processes occurring in the same reaction environment, such that RNA intermediates are rapidly converted to more stable DNA products. In some embodiments, linear amplification refers to amplification in which each amplification event proceeds through a transcription and/or reverse transcription step that does not involve thermocycling-driven exponential PCR amplification. In some embodiments, products may undergo additional rounds of transcription and reverse transcription while still avoiding the amplification bias associated with PCR.

As used herein, the term “nucleoside” refers to a molecule having a purine or pyrimidine base covalently linked to a ribose or deoxyribose sugar. Exemplary nucleosides include adenosine, guanosine, cytidine, uridine and thymidine. Additional exemplary nucleosides include inosine, 1-methyl inosine, pseudouridine, 5,6-dihydrouridine, ribothymidine, 2N-methylguanosine and 2,2N,N-dimethylguanosine (also referred to as “rare” nucleosides).

The term “nucleotide” refers to a nucleoside having one or more phosphate groups joined in ester linkages to the sugar moiety. Exemplary nucleotides include nucleoside monophosphates, diphosphates and triphosphates. The terms “polynucleotide,” “oligonucleotide” and “nucleic acid molecule” are used interchangeably herein and refer to a polymer of nucleotides, either deoxyribonucleotides or ribonucleotides, of any length joined together by a phosphodiester linkage between 5′ and 3′ carbon atoms. Polynucleotides can have any three-dimensional structure and can perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. The term also refers to both double-and single-stranded molecules. Unless otherwise specified or required, any embodiment of this disclosure that comprises a polynucleotide encompasses both the double-stranded form and each of two complementary single-stranded forms known or predicted to make up the double-stranded form. A polynucleotide is composed of a specific sequence of four nucleotide bases: adenine (A); cytosine (C); guanine (G); thymine (T); and uracil (U) for thymine when the polynucleotide is RNA. Thus, the term polynucleotide sequence is the alphabetical representation of a polynucleotide molecule.

A nucleic acid used in the disclosure can also include native or non-native bases. In this regard, a native deoxyribonucleic acid can have one or more bases selected from the group consisting of adenine, thymine, cytosine or guanine, and a ribonucleic acid can have one or more bases selected from the group consisting of uracil, adenine, cytosine or guanine. Exemplary non-native bases that can be included in a nucleic acid, whether having a native backbone or analog structure, include, without limitation, inosine, xanthine, hypoxanthine, isocytosine, isoguanine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 5-halouracil, 5-halocytosine, 5-propynyl uracil, 5-propynyl cytosine, 6-azauracil, 6-azocytosine, 6-azathymine, 5-fluorouracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. A particular embodiment can utilize isocytosine and isoguanine in a nucleic acid in order to reduce non-specific hybridization, as generally described in U.S. Pat. No. 5,681,702, incorporated herein in its entirety.

As used herein, the term “primer” generally includes an oligonucleotide, either natural or synthetic, that is capable, upon forming a duplex with a polynucleotide template, of acting as a point of initiation of nucleic acid synthesis, such as a sequencing primer, and being extended from its 3′ end along the template so that an extended duplex is formed. The sequence of nucleotides added during the extension process is determined by the sequence of the template polynucleotide. Usually, primers are extended by a DNA polymerase. Primers usually have a length in the range of between 3 to 36 nucleotides, also 5 to 24 nucleotides, also from 14 to 36 nucleotides. Primers within the scope of the disclosure include orthogonal primers, amplification primers, constructions primers, and the like. Pairs of primers can flank a sequence of interest or a set of sequences of interest. Primers and probes can be degenerate or quasi-degenerate in sequence. Primers within the scope of the present disclosure bind adjacent to a target sequence. A “primer” may be considered a short polynucleotide, generally with a free 3′-OH group that binds to a target or template potentially present in a sample of interest by hybridizing with the target, and thereafter promoting polymerization of a polynucleotide complementary to the target. Primers of the instant disclosure are comprised of nucleotides ranging from 17 to 30 nucleotides. In one aspect, the primer is at least 17 nucleotides, or alternatively, at least 18 nucleotides, or alternatively, at least 19 nucleotides, or alternatively, at least 20 nucleotides, or alternatively, at least 21 nucleotides, or alternatively, at least 22 nucleotides, or alternatively, at least 23 nucleotides, or alternatively, at least 24 nucleotides, or alternatively, at least 25 nucleotides, or alternatively, at least 26 nucleotides, or alternatively, at least 27 nucleotides, or alternatively, at least 28 nucleotides, or alternatively, at least 29 nucleotides, or alternatively, at least 30 nucleotides, or alternatively, at least 50 nucleotides, or alternatively, at least 75 nucleotides or alternatively, at least 100 nucleotides.

According to certain aspects, an exemplary transposon system is a Tn5 transposon system. Other useful transposon systems are known to those of skill in the art and include Tn3 transposon system (see Maekawa, T., Yanagihara, K., and Ohtsubo, E. (1996), A cell-free system of Tn3 transposition and transposition immunity, Genes Cells 1, 1007-1016), Tn7 transposon system (see Craig, N. L. (1991), Tn7: a target site-specific transposon, Mol. Microbiol. 5, 2569-2573), Tn 10 tranposon system (see Chalmers, R. et al. (2000), Complete nucleotide sequence of Tn10, J. Bacteriol 182, 2970-2972), Piggybac transposon system (see Li, X. et al. (2013), PiggyBac transposase tools for genome engineering, Proc. Natl. Acad. Sci. USA 110, E2279-2287), Sleeping beauty transposon system (see Ivies, Z. et al. (1997), Molecular reconstruction of Sleeping Beauty, a Tcl-like transposon from fish, and its transposition in human cells, Cell 91, 501-510), Tol2 transposon system (see Kawakami, K. (2007), Tol2: a versatile gene transfer vector in vertebrates, Genome Biol. 8 Suppl. 1, S7.)

According to certain aspects, an exemplary RNA polymerase is T7 RNA polymerase. Other useful RNA polymerases are known to those of skill in the art and include T3 RNA polymerase (see Jorgensen, E. D., Durbin, R. K., Risman, S. S., and McAllister, W. T. (1991) Specific contacts between the bacteriophage T3, T7, and SP6 RNA polymerases and their promoters, J. Biol. Chem. 266, 645-651, incorporated herein in its entirety), and SP6 RNA polymerase (see Melton, D. A., Krieg, P. A., Rebagliati, M. R., Maniatis, T., Zinn, K., and Green, M. R. (1984) Efficient in vitro synthesis of biologically active RNA and RNA hybridization probes from plasmids containing a bacteriophage SP6 promoter, Nucleic Acids Res. 12, 7035-7056, incorporated herein in its entirety).

As used herein, “Perturb-seq” refers to a high-throughput method that combines single-cell RNA sequencing with CRISPR-based genetic perturbations to enable pooled screening of gene function at single-cell resolution. In this approach, cells are transduced with a library of guide RNAs, each targeting a different gene or regulatory element, such that individual cells carry distinct perturbations. Following perturbation, single-cell RNA sequencing is performed to measure the transcriptional response of each cell. The identity of the perturbation in each cell is determined by capturing and sequencing the guide RNA sequence, which is typically integrated into the genome or delivered on a plasmid. By linking the perturbation identity to the transcriptional profile, Perturb-seq enables systematic exploration of how genetic changes influence cellular phenotypes. This method can be applied to study gene regulatory networks, identify gene functions, and investigate cellular responses to perturbations in diverse biological contexts.

As used herein, “a uniform size” refers to a feature sequence or amplified product having substantially the same length across multiple instances, such that length variation among the instances is less than about 10%, less than about 5%, or less than about 2% of the average length. For example, if a feature sequence has an average length of 500 base pairs, instances with lengths between 490 and 510 base pairs (±2%) would be considered to have a uniform size.

As used herein, “a narrow size distribution” refers to a feature sequence or amplified product having a length distribution in which the majority of instances fall within a defined range relative to the average length. In some embodiments, a narrow size distribution means that at least 80%, at least 90%, or at least 95% of the instances have lengths within about 20%, within about 10%, or within about 5% of the average length. For example, if a feature sequence has an average length of 500 base pairs, a narrow size distribution may mean that at least 90% of the instances have lengths between 450 and 550 base pairs (±10%).

As used herein, a “feature sequence” refers to a defined nucleic acid sequence of interest that is intended to be captured, amplified, or detected within a nucleic acid amplification or library preparation workflow. A feature sequence may be genomically encoded, present on an episomal element, or introduced through genetic modification. Feature sequences include, but are not limited to, guide RNA sequences, DNA barcode sequences, open reading frame sequences, overexpression construct sequences, reporter sequences, selection markers, and other engineered or naturally occurring nucleic acid elements. The length of a feature sequence may range from about 50 bases to about 10,000 bases, including intermediate lengths such as about 100 bases, about 200 bases, about 500 bases, about 1,000 bases, about 2,000 bases, about 5,000 bases, or any value therebetween. In some embodiments, the feature sequence has a uniform size or narrow size distribution across multiple instances, such that length-dependent biases in amplification or capture are reduced or minimized.

As used herein, “one pot reaction” refers to a reaction in which two or more enzymatic steps are carried out in the same reaction vessel and in the same reaction mixture without an intermediate purification, transfer, or isolation step between the enzymatic steps. In some embodiments, reagents for the different enzymatic steps are present together from the start of the reaction. In some embodiments, one or more reagents are added sequentially to the same reaction vessel, provided that the reaction products are not removed, purified, or transferred to a different vessel between steps.

As used herein, “one-step reaction” refers to a reaction workflow in which two or more enzymatic processes are performed in a single continuous reaction environment without an intervening purification, isolation, or separate setup step between the processes. A one-step reaction may occur in a one pot format and may include enzymatic processes that occur simultaneously, substantially simultaneously, or in overlapping time periods within the same reaction environment.

As used herein, the term “captureable” refers to a nucleic acid molecule or sequence that can be selectively isolated, enriched, or recovered within a library preparation or sequencing workflow through hybridization, enzymatic processing, or other sequence-specific binding interactions. A captureable sequence is one that is made accessible to capture reagents, such as oligonucleotide probes, primers, or solid-phase binding partners, through its conversion to a compatible form or through the inclusion of recognition elements that facilitate binding. In some embodiments, a DNA-encoded feature becomes captureable by generating an RNA transcript of the feature using an RNA polymerase, wherein the RNA transcript can then be captured using RNA-binding reagents or converted to cDNA for capture in a cDNA-based workflow. In some embodiments, a sequence is rendered captureable by incorporating an adapter, barcode, or primer binding site that permits selective amplification or enrichment. The term encompasses sequences that are captureable in their native form as well as sequences that are made captureable through enzymatic modification, such as transcription, reverse transcription, ligation, or tagmentation. A captureable sequence may be captured through direct binding to a solid support, through hybridization to a barcoded oligonucleotide, or through incorporation into an amplification product that is subsequently isolated or sequenced.

As used herein, an “RNA polymerase promoter” refers to a DNA sequence that is recognized and bound by an RNA polymerase enzyme, thereby initiating transcription of a downstream DNA sequence into RNA. The RNA polymerase promoter directs the RNA polymerase to begin RNA synthesis at a defined start site and in a specific orientation relative to the template DNA strand. RNA polymerase promoters include bacteriophage promoters such as T7, T3, and SP6 promoters, which are recognized by their cognate RNA polymerases and enable efficient in vitro transcription. The promoter sequence may be positioned immediately upstream of a gene, coding sequence, or other DNA region intended for transcription. In some embodiments, the RNA polymerase promoter is a T7 promoter, which is recognized by T7 RNA polymerase and initiates transcription in the 5′ to 3′ direction along the non-template strand. The promoter may be introduced into a DNA molecule through various methods, including adapter ligation, primer extension, transposase-mediated insertion, or PCR amplification using primers that contain the promoter sequence. The orientation of the promoter relative to the DNA sequence of interest determines the direction of transcription and the strand from which the RNA transcript is generated.

As used herein, a “reverse transcriptase primer binding region” refers to a DNA or RNA sequence that serves as a binding site for a primer oligonucleotide used in reverse transcription. The reverse transcriptase primer binding region provides a sequence complementary to a primer, allowing the primer to hybridize to the RNA transcript or template and serve as the initiation point for DNA synthesis by the reverse transcriptase enzyme. The primer binding region may be positioned at a defined location relative to the RNA polymerase promoter and the transcribed sequence, such that the primer anneals to the RNA transcript produced during in vitro transcription. In some embodiments, the reverse transcriptase primer binding region is located downstream of the RNA polymerase promoter and within or adjacent to the transcribed sequence. The primer binding region may correspond to a known sequence element, such as an adapter sequence, a poly(A) tail, a universal priming site, or a feature-specific sequence. The reverse transcriptase primer may be designed to hybridize specifically to the primer binding region, enabling selective reverse transcription of the intended RNA transcript. In some embodiments, the primer binding region is incorporated into the DNA molecule during library preparation, tagmentation, or adapter ligation steps. The primer binding region may be the same for multiple templates in a library, enabling uniform priming and reverse transcription across diverse sequences, or may be feature-specific to enable selective amplification of particular targets.

As used herein, a “molecular barcode” or “barcode” refers to a nucleic acid sequence tag that serves to identify a molecule, partition, bead, cell, or sample of origin. The molecular barcode may be a defined sequence of nucleotides, typically between about 4 and about 30 nucleotides in length, that is attached to or associated with a nucleic acid molecule or other entity to enable tracking, identification, or quantification of that entity throughout a workflow. Molecular barcodes may be used to distinguish individual molecules within a sample, to link nucleic acids to the cell or partition from which they originated, to identify different samples in a multiplexed reaction, or to track the origin of amplification products. In some embodiments, a molecular barcode is incorporated into a nucleic acid molecule during library preparation, such as through ligation, primer extension, or transposase-mediated insertion. In some embodiments, the molecular barcode is part of an adapter sequence or primer that is added to the nucleic acid molecule. In some embodiments, the molecular barcode is a unique molecular identifier (UMI) that labels individual molecules prior to amplification, enabling downstream correction for amplification bias or quantification of original molecule abundance. In some embodiments, the molecular barcode is a cell barcode that identifies the cell or partition from which a nucleic acid originated in a single-cell sequencing workflow. In some embodiments, the molecular barcode is a sample barcode or index that allows multiple samples to be pooled and sequenced together, with the barcode enabling demultiplexing and assignment of sequence reads to the originating sample. Molecular barcodes may be read during sequencing to provide information about the identity or origin of the associated nucleic acid. The barcode sequence may be designed to minimize sequence similarity between different barcodes, such as by maintaining a minimum Hamming distance, to reduce the likelihood of misassignment due to sequencing errors.

As used herein, an “abortive RNA transcript” refers to a short RNA molecule that is produced when an RNA polymerase initiates transcription but dissociates from the DNA template before completing synthesis of a full-length transcript. Abortive transcripts typically range in length from about 2 nucleotides to about 15 nucleotides, though they may be longer depending on the polymerase and reaction conditions. These short RNA fragments result from premature termination of transcription during the early stages of elongation, before the polymerase has formed a stable elongation complex with the DNA template. Abortive transcription is a normal occurrence during in vitro transcription reactions, particularly with bacteriophage RNA polymerases such as T7 RNA polymerase. The production of abortive transcripts can represent a substantial fraction of the RNA polymerase activity in the early stages of transcription. In the context of the integrated transcription and reverse transcription (ITART) method disclosed herein, abortive RNA transcripts can serve as unintended primers for reverse transcription in the absence of a designed reverse transcriptase primer. When a reverse transcriptase enzyme is present in the same reaction, abortive transcripts may hybridize to off-target sites on RNA or DNA molecules and initiate synthesis of complementary DNA from those sites, leading to background amplification products. The length and sequence composition of abortive transcripts depend on the promoter sequence, the RNA polymerase used, and the reaction conditions such as nucleotide concentrations and temperature.

As used herein, a “blocking oligonucleotide” refers to a short synthetic nucleic acid molecule that is designed to hybridize to a specific target sequence, thereby preventing that target sequence from participating in unwanted reactions or interactions. Blocking oligonucleotides are typically single-stranded DNA or RNA molecules, though modified nucleic acids such as locked nucleic acids (LNAs), peptide nucleic acids (PNAs), or phosphorothioate-modified oligonucleotides may also be used. The length of a blocking oligonucleotide may range from about 8 nucleotides to about 50 nucleotides, including intermediate lengths such as about 10 nucleotides, about 15 nucleotides, about 20 nucleotides, about 25 nucleotides, about 30 nucleotides, or any value therebetween. In the context of the present disclosure, blocking oligonucleotides are used to hybridize to abortive RNA transcripts produced during in vitro transcription, thereby preventing the abortive transcripts from serving as primers for reverse transcription. By binding to the abortive transcripts, the blocking oligonucleotides sequester the abortive transcripts and prevent them from initiating off-target reverse transcription reactions. The blocking oligonucleotides may be designed to have a sequence complementary to the expected sequence of abortive transcripts, taking into account the promoter sequence and the nucleotide composition of the early transcribed region. In some embodiments, blocking oligonucleotides may be modified to enhance binding affinity or stability, such as through the incorporation of LNA bases, 2′-O-methyl modifications, or other chemical modifications that increase hybridization strength or nuclease resistance. In some embodiments, blocking oligonucleotides may be designed to lack a 3′-hydroxyl group, such as through the addition of a 3′-phosphate, a 3′-amino group, or a 3′-dideoxy modification, thereby preventing the blocking oligonucleotide itself from being extended by a polymerase. The use of blocking oligonucleotides in the ITART method reduces background signal from off-target reverse transcription and improves the specificity of the amplification reaction.

As used herein, the term “isothermal” refers to a reaction or process that is carried out at a substantially constant temperature throughout the duration of the reaction, without requiring thermal cycling or repeated heating and cooling steps. An isothermal reaction may be performed at a single defined temperature or within a narrow temperature range that does not involve deliberate temperature changes designed to denature nucleic acids, anneal primers, or activate temperature-dependent enzymatic steps. In some embodiments, an isothermal reaction is maintained at a temperature between about 20 degrees Celsius and about 65 degrees Celsius. In some embodiments, the temperature variation during the isothermal reaction is less than about 10 degrees Celsius, less than about 5 degrees Celsius, less than about 3 degrees Celsius, or less than about 2 degrees Celsius.

### MODES OF CARRYING OUT THE DISCLOSURE

### Isothermal Methods for Amplifying a Dna Molecule

An aspect of the disclosure is directed to an isothermal method for amplifying a DNA molecule in a sample comprising (a) producing an RNA copy of the DNA molecule using an RNA polymerase, and (b) producing a complementary DNA (cDNA) copy of the RNA copy using a reverse transcriptase, thereby linearly amplifying the DNA molecule, wherein (a) and (b) are carried out as a one pot reaction.

In some embodiments, the RNA polymerase and the reverse transcriptase are active at the same time. In some embodiments, the RNA polymerase and the reverse transcriptase work simultaneously. In some embodiments, the RNA polymerase and the reverse transcriptase work substantially simultaneously. In some embodiments, the reverse transcriptase creates a DNA copy of the RNA produced by the RNA polymerase simultaneously. In some embodiments, the reverse transcriptase creates a DNA copy of the RNA produced by the RNA polymerase substantially simultaneously.

In some embodiments, the sample comprises a biological sample or an environmental sample.

In some embodiments, reverse transcribing the RNA copy into the cDNA copy is achieved while the RNA copy is being produced.

In some embodiments, the DNA molecule comprises an RNA polymerase promoter and a reverse transcriptase primer binding region.

According to one aspect, the DNA molecule includes an RNA polymerase promoter sequence and an RNA polymerase is used to make RNA amplicons from the DNA fragment templates and a reverse transcriptase to make single-stranded DNA from the RNA amplicons. Complements may then be made to the single-stranded DNA to make double-stranded DNA, resulting in amplicons linearly amplified from the original DNA fragment template.

In some embodiments, prior to the RNA transcription and reverse transcription, the DNA molecule is modified to contain an RNA polymerase promoter and a reverse transcriptase primer binding region. In some embodiments, the RNA polymerase promoter and the reverse transcriptase primer binding region are added to the DNA molecule using a transposase. In some embodiments, the transposase is selected from a Tn5 transposase, a Tn3 transposase, a Tn7 transposase, a Tn10 transposase, a PiggyBac transposase, a Sleeping Beauty transposase, or a Tol2 transposase. In some embodiments, the RNA polymerase promoter is introduced by an adapter, a primer, or a transposase.

In some embodiments, the RNA polymerase promoter is a unidirectional promoter or a bidirectional promoter. In some embodiments, the RNA polymerase promoter is a T7 promoter, a T3 promoter, an SP6 promoter, a T7lac promoter, or another bacteriophage or bacterial promoter that can be recognized by an RNA polymerase in vitro.

In some embodiments, the reaction buffer comprises a ribonucleotide triphosphate (NTP) to deoxyribonucleotide triphosphate (dNTP) ratio (NTP: dNTP) of between 5:1 and 1:2 (e.g., 5:1, 5:2, 5:3, 5:4, 1:1, 5:6, 5:7, 5:8, 5:9, or 1:2, or any ratio therebetween). In some embodiments, the NTP: dNTP ratio is 1:1.

In some embodiments, the reaction buffer comprises between 2-5 mM (e.g., 2, 2.5, 3, 3.5, 4, 4.5, 5 mM, or any value therebetween) Mg²⁺.

In some embodiments, the reaction buffer comprises between 0.5-10 mM (e.g., 0.5, 1, 1.5, 2, 2.5, 3, 3.5, 4, 4.5, 5, 5.5, 6, 6.5, 7, 7.5, 8, 8.5, 9, 9.5, 10 mM, or any value therebetween) Mg²⁺.

In some embodiments, the DNA molecule further comprises at least one molecular barcode. In some embodiments, the molecular barcode identifies a partition or a cell of origin.

In some embodiments, the RNA polymerase is selected from a T7 RNA polymerase, a T3 RNA polymerase, or an SP6 RNA polymerase.

In some embodiments, the reverse transcriptase lacks RNase activity and/or exonuclease activity. In some embodiments, the reverse transcriptase exhibits activity in a reaction buffer containing both ribonucleotide triphosphates and deoxyribonucleotide triphosphates. In some embodiments, the reverse transcriptase is compatible with a magnesium concentration in the reaction buffer that also supports RNA polymerase activity. In some embodiments, the reverse transcriptase has low RNase H activity or is an RNase H-minus variant. In some embodiments, the reverse transcriptase is operational at isothermal temperatures that overlap with the operational temperature range of the RNA polymerase. In some embodiments, the reverse transcriptase maintains activity at a constant temperature between about 20 degrees Celsius and about 65 degrees Celsius. In some embodiments, the reverse transcriptase maintains activity at about 37 degrees Celsius. In some embodiments, the reverse transcriptase is selected such that it can function in the same reaction buffer as the RNA polymerase without requiring separate buffer conditions or temperature adjustments.

In some embodiments, the reverse transcriptase is selected from a Maxima™ H-Minus reverse transcriptase, an HIV reverse transcriptase, avian myeloblastosis virus (AMV) reverse transcriptase, a Moloney murine leukemia virus (M-MuLV or MMLV) based reverse transcriptase (as described in Yang, Youhui, et al., Bioresources and Bioprocessing 12.1 (2025): 12, which is incorporated herein in its entirety), or a Group II intron reverse transcriptase (as described in Mohr, Sabine, et al., Rna 19.7(2013 ): 958-970, incorporated herein in its entirety).

In some embodiments, abortive RNA transcripts from the IVT reaction are contacted with one or more blocking oligonucleotides. In some embodiments, the one or more blocking oligonucleotides hybridize to abortive RNA transcripts produced during in vitro transcription, thereby reducing reverse transcription initiated from the abortive RNA transcripts.

In some embodiments, the DNA molecule comprises genomic DNA or cDNA. In some embodiments, the DNA molecule is obtained from a single cell or a small population of cells.

In some embodiments, amplification is achieved at a constant temperature between about 20 and about 65 degrees Celsius (e.g., about 20, 25, 30, 35, 40, 45, 50, 55, 60, 65 degrees Celsius or any value therebetween). In some embodiments, amplification is achieved at a constant temperature between about 25 and about 55 degrees Celsius (e.g., about 25, 30, 35, 40, 45, 50, 55 degrees Celsius or any value therebetween). In some embodiments, amplification is achieved at a constant temperature between about 30 and about 50 degrees Celsius (e.g., about 30, 35, 40, 45, 50 degrees Celsius or any value therebetween). In some embodiments, amplification is achieved at a constant temperature between about 35 and about 45 degrees Celsius (e.g., about 35, 40, 45 degrees Celsius or any value therebetween). In some embodiments, amplification is achieved at a constant temperature between about 30 and about 40 degrees Celsius (e.g., about 30, 35, 40 degrees Celsius or any value therebetween). In some embodiments, the amplifying is achieved at about 37 degrees Celsius (e.g., 35, 35.5, 36, 36.5, 37, 37.5, 38, 38.5, 39 degrees Celsius or any value therebetween).

In some embodiments, the linearly amplified DNA molecule is used directly in a sequencing workflow. In some embodiments, the linearly amplified DNA molecule is used in the sequencing workflow without a PCR amplification step. In some embodiments, the linearly amplified DNA molecule is converted to double-stranded DNA for use in a sequencing workflow. In some embodiments, the linearly amplified DNA molecule is further amplified using a polymerase chain reaction (PCR) with less than 20 cycles (e.g., 19, 18, 17, 16, 15, 14, 13, 12, 11, 10 or 9 cycles).

In some embodiments, the cDNA copy comprises the RNA polymerase promoter sequence.

In some embodiments, the cDNA copy is used as a template for producing a second RNA copy using the RNA polymerase, and producing a second complementary DNA copy of the second RNA copy using the reverse transcriptase, wherein producing the second RNA copy and producing the second complementary DNA copy are carried out in the same one pot reaction.

In some embodiments, the second RNA copy is produced from the RNA polymerase promoter sequence present in the cDNA copy.

In some embodiments, the RNA polymerase recognizes the RNA polymerase promoter sequence in the cDNA copy and initiates transcription to produce the second RNA copy.

In some embodiments, the RNA polymerase promoter sequence is transcribed as part of the RNA copy and is present in the cDNA copy following reverse transcription.

In some embodiments, the cDNA copy undergoes multiple rounds of transcription and reverse transcription within the same one pot reaction, thereby achieving iterative linear amplification.

### Methods for Preparing a Sequencing Library

An aspect of the disclosure is directed to an isothermal method for preparing a sequencing library using the ITART methodology as described herein. In some embodiments, the isothermal method for preparing a sequencing library from a partitioned sample, comprises (a) providing in a partition a nucleic acid molecule comprising a feature sequence and an RNA polymerase promoter upstream of the feature sequence, (b) producing an RNA transcript of the feature sequence using an RNA polymerase, and (c) producing a complementary DNA (cDNA) copy of the RNA transcript using a reverse transcriptase, wherein (b) and (c) are carried out as a one pot reaction, thereby amplifying and capturing the feature sequence in the partition.

In some embodiments, the partitioned sample comprises a single-cell sequencing library. The single-cell sequencing library may be derived from cells that have been isolated, compartmentalized, or otherwise processed to enable single-cell analysis. The library may contain nucleic acids from individual cells, wherein each cell or partition is associated with a unique identifier, such as a barcode, that links the nucleic acids to the originating cell or partition. The single-cell sequencing library may be prepared using methods known in the art, including droplet-based encapsulation, microwell-based partitioning, or other microfluidic techniques that enable high-throughput processing of individual cells. The library may include RNA transcripts, cDNA derived from RNA transcripts, genomic DNA, or other nucleic acid species present in the cell. In some embodiments, the single-cell sequencing library includes cells that have been perturbed, such as through CRISPR-based gene editing, RNA interference, chemical treatment, or other perturbation methods, and the library preparation workflow is designed to capture both the perturbation identity and the transcriptional or genomic state of the cell. The single-cell sequencing library may further include engineered features, such as guide RNA sequences, DNA barcodes, overexpression constructs, or other nucleic acid elements that have been introduced into the cells through transduction, transfection, or other delivery methods. The library may be prepared in a format compatible with next-generation sequencing platforms, including short-read sequencing, long-read sequencing, or single-molecule sequencing technologies.

In some embodiments, the partition is a droplet, emulsion, encapsulation, microwell, or other compartment that preserves an association between the feature sequence and a cell or partition of origin. The partition may be formed through microfluidic encapsulation, in which individual cells are isolated within aqueous droplets surrounded by an immiscible oil phase. The partition may be formed through emulsion-based methods, in which cells are distributed into discrete reaction volumes within a continuous phase. The partition may be an encapsulation structure, such as a hydrogel bead or polymer capsule, that physically confines the cell and associated reagents. The partition may be a microwell, such as a feature etched or molded into a substrate, in which a single cell or a defined number of cells are deposited. The partition may be any other compartment that spatially separates individual cells or small groups of cells, thereby preventing cross-contamination of nucleic acids or other cellular material between different cells. The partition preserves the association between the feature sequence and the originating cell or partition throughout the reaction and downstream processing steps. In workflows involving barcoding, the partition allows the feature sequence to be linked to a unique barcode that identifies the originating cell or partition. The partition may contain reagents for cell lysis, nucleic acid capture, amplification, and library preparation. The partition may be stable throughout the amplification reaction and may be maintained until the feature sequence has been captured and associated with a cell-specific identifier. In some embodiments, the partition is maintained until sequencing library preparation is complete. In some embodiments, the partition is disrupted or pooled after the feature sequence has been captured and linked to a barcode, allowing downstream processing of the combined library.

In some embodiments, the RNA polymerase and the reverse transcriptase are active at the same time, and (b) and (c) occur substantially simultaneously.

In some embodiments, steps (b) and (c) are carried out at a constant temperature.

In some embodiments, the feature sequence is genomically encoded.

In some embodiments, the feature sequence is not natively transcribed prior to step (b).

In some embodiments, the RNA polymerase promoter is a T7 promoter, a T3 promoter, an SP6 promoter, a T7lac promoter, or another bacteriophage or bacterial promoter that can be recognized by an RNA polymerase in vitro.

In some embodiments, the feature sequence is selected from a guide RNA sequence, a DNA barcode sequence, an open reading frame sequence, an overexpression construct sequence, or a sequence of interest.

In some embodiments, the method further comprises hybridizing a feature sequence-specific primer to the RNA transcript or the cDNA copy to enrich a region of interest downstream of the RNA polymerase promoter.

In some embodiments, the sequencing library further comprises native RNA from the partition, and the method captures the feature sequence together with the native RNA.

In some embodiments, the partition comprises a barcode such that the amplified and captured feature sequence remains associated with the cell or partition of origin.

In some embodiments, the feature sequence has a uniform size or a narrow size distribution across a plurality of partitions. In some embodiments, the feature sequence has a length between about 50 bases and about 10,000 bases. In some embodiments, the feature sequence has a length between about 100 bases and about 5,000 bases. In some embodiments, the feature sequence has a length between about 200 bases and about 2,000 bases. In some embodiments, the feature sequence has a length between about 300 bases and about 1,000 bases. In some embodiments, the feature sequence has a length of about 50 bases, about 100 bases, about 200 bases, about 300 bases, about 400 bases, about 500 bases, about 600 bases, about 700 bases, about 800 bases, about 900 bases, about 1,000 bases, about 1,500 bases, about 2,000 bases, about 2,500 bases, about 3,000 bases, about 4,000 bases, about 5,000 bases, about 6,000 bases, about 7,000 bases, about 8,000 bases, about 9,000 bases, about 10,000 bases, or any value therebetween.

In some embodiments, the method is used in a single-cell CRISPR screening workflow. In some embodiments, the single-cell CRISPR screening workflow is a Perturb-seq workflow. In some embodiments, the feature sequence comprises a guide RNA sequence. The guide RNA sequence may be part of a CRISPR perturbation construct, wherein the guide RNA directs a Cas nuclease to a specific genomic target for gene editing or transcriptional modulation. In some embodiments, the guide RNA sequence is delivered to the cell by lentiviral transduction, electroporation, or another nucleic acid delivery method. In some embodiments, the guide RNA sequence is stably integrated into the genome of the cell. In some embodiments, the guide RNA sequence is expressed from a plasmid or other episomal element. In some embodiments, the guide RNA sequence is positioned downstream of an RNA polymerase promoter such that in vitro transcription produces an RNA transcript corresponding to the guide RNA sequence. In some embodiments, the guide RNA sequence has a length between about 17 nucleotides and about 24 nucleotides. In some embodiments, the guide RNA sequence is flanked by adapter sequences that facilitate capture and sequencing. In some embodiments, the guide RNA sequence is linked to a barcode that identifies the originating cell or partition. In some embodiments, multiple different guide RNA sequences are present in a pooled library, and each cell or partition receives one or a small number of guide RNA sequences. In some embodiments, the guide RNA sequence is captured and sequenced to determine which perturbation was present in a given cell or partition. In some embodiments, the method reduces cross-cell contamination and improves the accuracy of guide RNA assignment compared to conventional PCR-based amplification methods.

### Methods for Capturing a Feature Sequence

An aspect of the disclosure is directed to a method for capturing a feature sequence in an RNA-based library preparation workflow, comprising providing a DNA molecule comprising an RNA polymerase promoter upstream of a feature sequence, producing an RNA transcript of the feature sequence using an RNA polymerase, and producing a complementary DNA (cDNA) copy of the RNA transcript using a reverse transcriptase, wherein the producing an RNA transcript and the producing a cDNA copy are carried out as a one pot isothermal reaction, thereby rendering the feature sequence captureable in the RNA-based library preparation workflow.

In some embodiments, the feature sequence is not natively transcribed.

In some embodiments, the feature sequence is selected from a guide RNA sequence, a DNA barcode sequence, an open reading frame sequence, an overexpression construct sequence, or a sequence of interest.

In some embodiments, a feature sequence-specific primer is used to enrich a region of interest downstream of the RNA polymerase promoter.

In some embodiments, the RNA polymerase promoter is a T7 promoter, a T3 promoter, an SP6 promoter, a T7lac promoter, or another bacteriophage or bacterial promoter that can be recognized by an RNA polymerase in vitro

In some embodiments, the RNA transcript and the cDNA copy are generated in a partition.

In some embodiments, the partition is associated with a single cell.

In some embodiments, the RNA transcript and the cDNA copy are generated in a bulk reaction.

In some embodiments, the cDNA copy comprises the RNA polymerase promoter sequence.

In some embodiments, the cDNA copy is used as a template for producing a second RNA transcript using the RNA polymerase, and producing a second complementary DNA copy of the second RNA transcript using the reverse transcriptase, wherein producing the second RNA transcript and producing the second complementary DNA copy are carried out in the same one pot isothermal reaction.

In some embodiments, the feature sequence undergoes iterative linear amplification through multiple rounds of transcription and reverse transcription.

In some embodiments, the RNA polymerase promoter sequence is encoded in the RNA transcript and is present in the cDNA copy following reverse transcription.

### Compositions and Kits

Another aspect of the disclosure is directed to a composition comprising an RNA polymerase and a reverse transcriptase in the same reaction buffer, wherein the RNA polymerase and the reverse transcriptase are active in the reaction buffer at the same time.

In some embodiments, the RNA polymerase is selected from a T7 RNA polymerase, a T3 RNA polymerase, or an SP6 RNA polymerase.

In some embodiments, the reverse transcriptase is selected from a Maxima™ H-Minus™ reverse transcriptase (e.g., Thermo Fisher® Catalog No: EP0751), an HIV reverse transcriptase, avian myeloblastosis virus (AMV) reverse transcriptase, a Moloney murine leukemia virus (M-MuLV or MMLV) based reverse transcriptase, or a Group II intron reverse transcriptase.

In some embodiments, the composition further comprises one or more primers configured to hybridize to a feature sequence transcribed from a DNA molecule comprising an RNA polymerase promoter upstream of the feature sequence. In some embodiments, the composition further comprises one or more blocking oligonucleotides configured to hybridize to abortive RNA transcripts.

In some embodiments, the composition further comprises one or more salts to maintain ionic strength and support enzyme stability. The salts may include potassium chloride, sodium chloride, ammonium sulfate, or combinations thereof. The concentration of salts in the reaction buffer may be between about 10 mM and about 200 mM, including intermediate concentrations such as about 25 mM, about 50 mM, about 75 mM, about 100 mM, about 125 mM, about 150 mM, about 175 mM, or any value therebetween. In some embodiments, the salt concentration is selected to optimize the activity of both the RNA polymerase and the reverse transcriptase in the same reaction environment. Alternative embodiments may include the use of mixed salt systems, where different salts are combined to achieve desired ionic conditions, or the use of buffer systems such as Tris-HCl, HEPES, or phosphate-buffered saline that inherently provide ionic strength.

In some embodiments, the composition further comprises one or more reducing agents to protect enzyme active sites and maintain proper redox conditions. The reducing agents may include dithiothreitol (DTT), beta-mercaptoethanol, tris(2-carboxyethyl)phosphine (TCEP), or combinations thereof. The concentration of the reducing agent may be between about 0.1 mM and about 10 mM, including intermediate concentrations such as about 0.5 mM, about 1 mM, about 2 mM, about 5 mM, or any value therebetween. The reducing agent may be included to prevent oxidation of cysteine residues in the enzymes, thereby maintaining enzyme activity over extended reaction times. Alternative embodiments may employ alternative reducing agents or omit reducing agents in cases where the enzymes are stable under oxidative conditions or where the reaction is performed under anaerobic conditions.

In some embodiments, the composition further comprises one or more stabilizers to enhance enzyme stability during storage and reaction. The stabilizers may include glycerol, trehalose, sorbitol, bovine serum albumin (BSA), polyethylene glycol (PEG), or combinations thereof. The concentration of the stabilizer may be between about 1% and about 50% (v/v or w/v), including intermediate concentrations such as about 5%, about 10%, about 15%, about 20%, about 25%, about 30%, about 40%, or any value therebetween. Glycerol is commonly used at concentrations between about 10% and about 50% to stabilize enzymes during storage. Trehalose may be included at concentrations between about 1% and about 10% to protect enzymes during lyophilization or freeze-thaw cycles. BSA may be included at concentrations between about 0.01 mg/mL and about 1 mg/mL to prevent enzyme adsorption to tube walls and to stabilize enzyme activity. Alternative embodiments may employ combinations of stabilizers or use alternative stabilizing agents such as polyvinylpyrrolidone or other polymers.

In some embodiments, the composition further comprises one or more RNase inhibitors to protect RNA intermediates from degradation. The RNase inhibitors may include recombinant RNase inhibitors, such as those derived from human or porcine sources, or chemical inhibitors such as ribonucleoside-vanadyl complexes. The concentration of the RNase inhibitor may be between about 0.1 units per microliter and about 10 units per microliter, including intermediate concentrations such as about 0.5 units per microliter, about 1 unit per microliter, about 2 units per microliter, about 5 units per microliter, or any value therebetween. The inclusion of RNase inhibitors may be beneficial in applications where RNA stability is a concern, such as when working with low-input samples or when reaction times are extended. Alternative embodiments may employ RNase inhibitors with different specificities or may omit RNase inhibitors in cases where the reverse transcriptase rapidly converts RNA to DNA, thereby minimizing the opportunity for RNA degradation.

In some embodiments, the composition further comprises one or more crowding agents to enhance enzyme activity and reaction efficiency. The crowding agents may include polyethylene glycol (PEG), Ficoll, dextran, or combinations thereof. The concentration of the crowding agent may be between about 1% and about 20% (w/v), including intermediate concentrations such as about 2%, about 5%, about 10%, about 15%, or any value therebetween. Crowding agents mimic the crowded intracellular environment and may increase the effective concentration of reactants, thereby enhancing reaction rates and yields. PEG may be used at molecular weights ranging from about 200 Da to about 20,000 Da, with PEG 8000 being commonly employed at concentrations between about 5% and about 15%. Ficoll may be used at concentrations between about 5% and about 15%. Alternative embodiments may employ different molecular weight PEG species or combinations of crowding agents to optimize reaction conditions for specific enzyme pairs or reaction formats.

In some embodiments, the reaction buffer is maintained at a pH between about 6.0 and about 9.0. In some embodiments, the pH is between about 6.5 and about 8.5. In some embodiments, the pH is between about 7.0 and about 8.0. In some embodiments, the pH is about 7.0, about 7.5, about 8.0, or any value therebetween. The pH may be maintained using a buffering agent such as Tris-HCl, HEPES, MOPS, phosphate buffer, or combinations thereof. The buffering agent may be present at a concentration between about 10 mM and about 100 mM, including intermediate concentrations such as about 20 mM, about 50 mM, about 75 mM, or any value therebetween. The optimal pH may depend on the specific RNA polymerase and reverse transcriptase employed, and may be adjusted to balance the activity of both enzymes in the same reaction. Alternative embodiments may employ different buffering systems or pH ranges depending on the enzyme pair and application requirements.

In some embodiments, the composition is provided in a storage format suitable for long-term stability and ease of use. The composition may be provided as a single-tube formulation, in which all components are pre-mixed in a single container. The composition may be provided as a lyophilized or freeze-dried formulation, in which the enzymes and other components are dried and reconstituted with water or buffer before use. Lyophilization may enhance storage stability and extend shelf life. The composition may be provided as a multi-component kit, in which the enzymes, reaction buffer, and other reagents are packaged separately and combined by the user prior to use. In some embodiments, the RNA polymerase and reverse transcriptase are pre-mixed in the same reaction buffer and provided as a single ready-to-use solution. In other embodiments, the RNA polymerase and reverse transcriptase are packaged separately and combined by the user at the time of use, allowing for greater flexibility in optimizing enzyme ratios or reaction conditions. Alternative embodiments may include storage formats such as frozen aliquots, stabilized liquid formulations, or components immobilized on solid supports.

Another aspect of the disclosure is directed to a kit comprising the composition of the disclosure and instructions for use. In some embodiments, the instructions are for use in a partition-based single-cell sequencing workflow. In some embodiments, the instructions are for use in a bulk sequencing workflow. In some embodiments, the instructions are for capturing a guide RNA sequence, a DNA barcode sequence, an open reading frame sequence, an overexpression construct sequence, or a sequence of interest.

### EXAMPLES

Example 1-Extended Time IVT Required for low-input Template, Loses Most of the RNA Before cDNA Conversion by Following Reverse Transcription

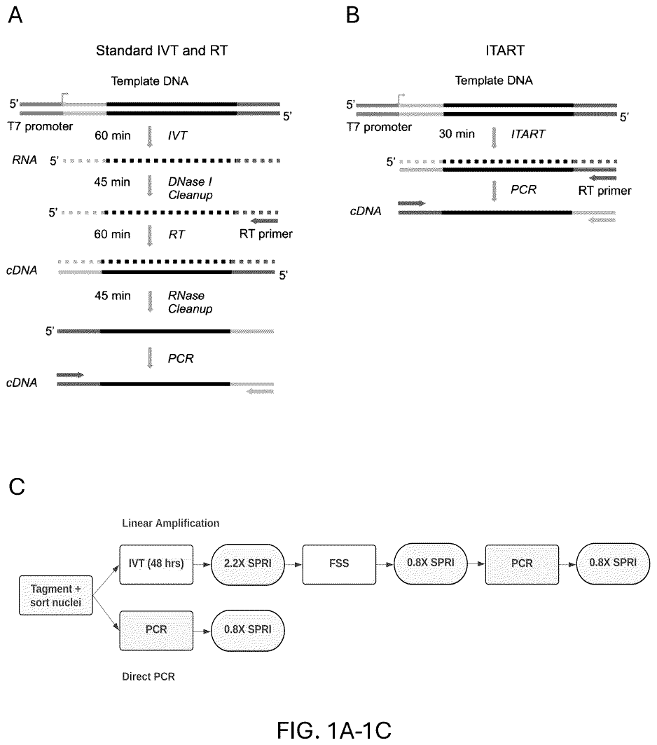

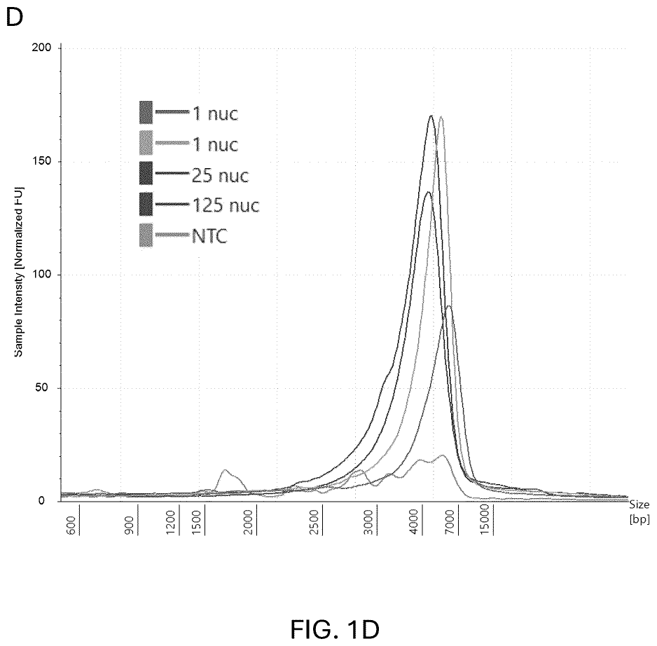

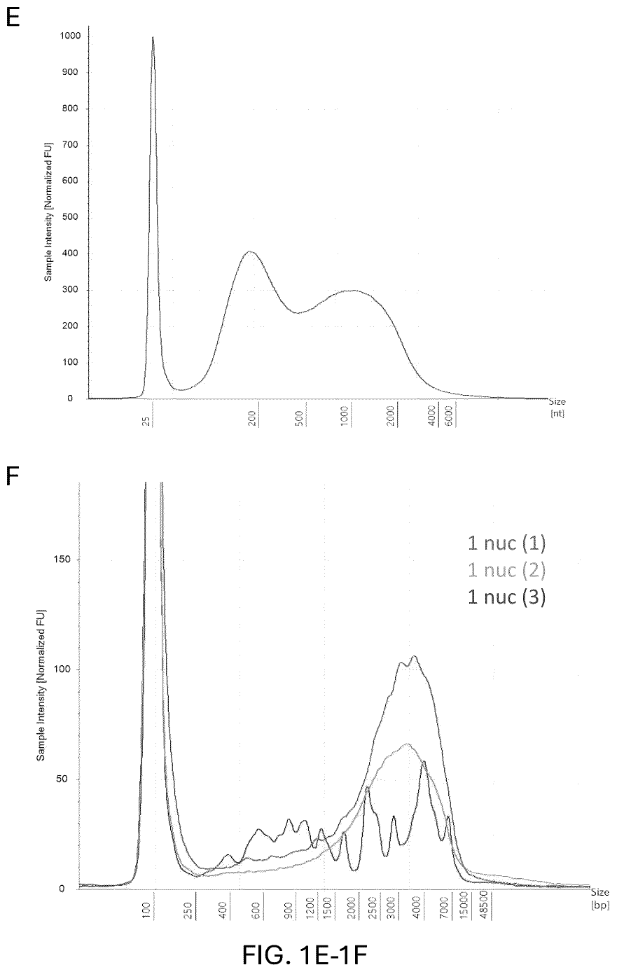

In one embodiment, ITART includes the simultaneous use of the T7 RNA polymerase and RTase, as well as a specific composition of the reaction buffer. Combining multiple processes in a single tube enhances the yield and quality of cDNA, which can be sequenced directly or amplified via additional PCR. Moreover, IVT requires template degradation and cleaning steps that can negatively impact cDNA yield. These steps are omitted in ITART (FIGS. 1A-1B).

The ITART method employs linear amplification of the targeted DNA region, reducing known biases caused by exponential amplification. PCR amplification can also cause strand-swapping between molecules, resulting in chimeric amplicons. For applications such as single-cell transcriptomics, ITART can provide quick amplification while reducing the rate of chimeric amplicon formation.

Polymerase Chain Reaction (PCR) provides robust and exponential amplification of DNA molecules, but has certain drawbacks including bias in amplification and replication errors that accumulate in high number of cycles. PCR is also ineffective and highly biased for amplifying low-quality (e.g., partially degraded) or low-abundance templates. In order to overcome these drawbacks, methods that use in vitro transcription (IVT) followed by reverse transcription (RT) have been developed. In these methods, IVT and RT reactions are performed as separate reactions, with the RT reaction following the IVT reaction. The IVT reaction takes over 48 hours from a low input template (FIG. 1C). Applicant observed that the RNA and subsequent cDNA obtained from these methods are of lower quality and most of the products were not at the expected correct size (FIGS. 1E-1F) as compared to direct PCR from sorted nuclei (FIG. 1D).

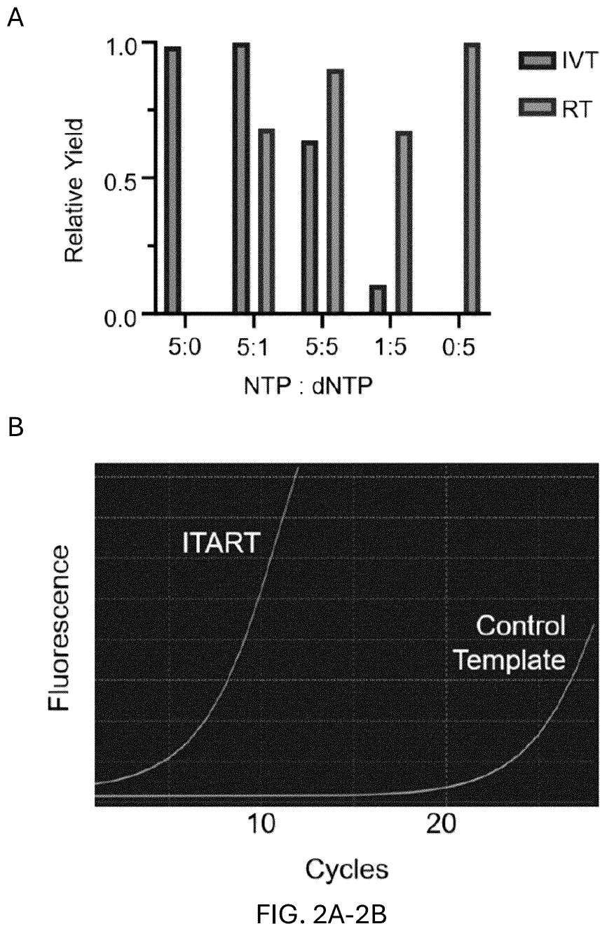

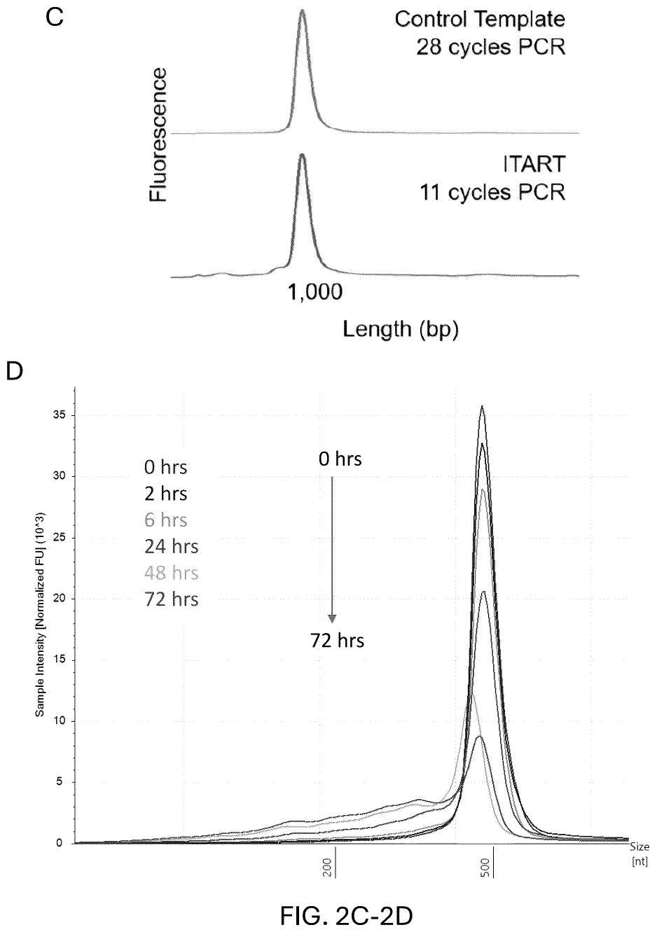

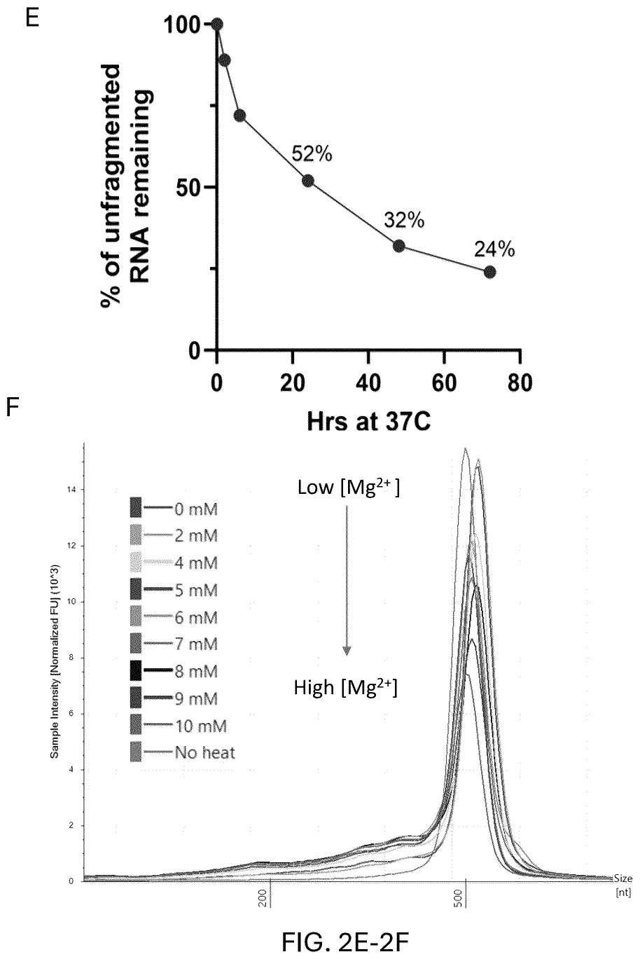

Applicant further tested the efficiencies of T7 RNA Polymerase and RTase in the reaction buffer with NTP and dNTP (FIG. 2A), and the amplification of ITART cDNA (FIGS. 2B-2C).

Applicant further observed that RNA produced by the IVT reaction is highly susceptible to degradation during the long reaction time and presence of high levels of Magnesium (4 mM or more) in these reactions (FIGS. 2D-2F). First, Applicant incubated RNA obtained from an IVT reaction in a buffer containing 4 mM Mg²⁺ for 0, 2, 6, 24, 48 or 72 hours at 37° C. and measured the amount of RNA after each incubation. Applicant observed that the concentration of RNA at the correct size was decreased over time (FIG. 2D). Applicant further found that half of the RNA was degraded in 24 hours, and by 48 hours, only 32% of the RNA was left intact (FIG. 2E). Applicant also tested different amounts of magnesium in buffer for a 24 hour incubation at 37° C., and observed that high concentration of magnesium increased RNA degradation (FIG. 2F).

### Example 2: The ITART Method

In order to overcome the disadvantages of the conventional methods, Applicant developed a method called “Integrated Transcription and Reverse Transcription (ITART).” ITART overcomes the disadvantages of IVT followed by RT by combining IVT and RT in a highly efficient one pot and one-step linear amplification. In this reaction, RT rapidly converts the de novo synthesized IVT RNA into more stable cDNA (FIG. 3A). The ITART method is suitable for DNA amplification workflow steps in genomic and transcriptomic assays such as single-cell genome sequencing, single-cell transcriptomics, RNA-seq, transposon-based techniques, and multiplex protein analysis via proximity extension assays.

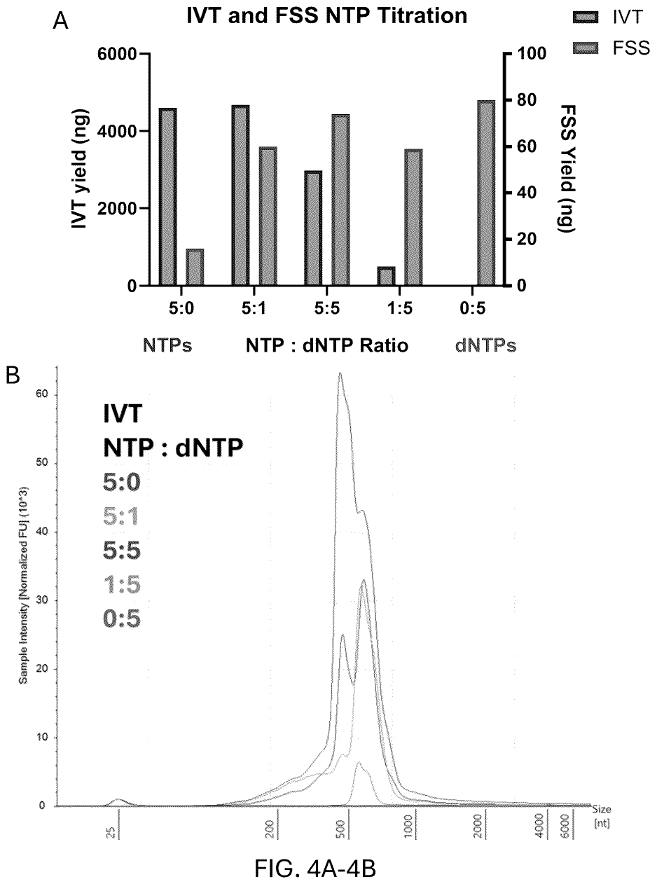

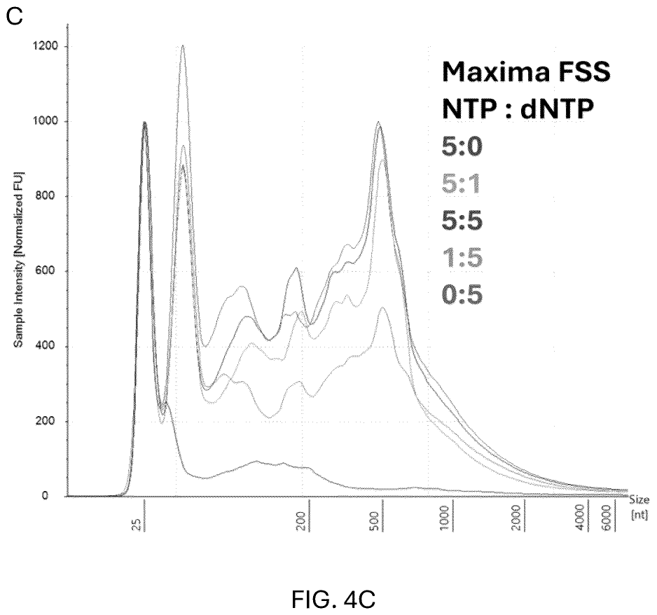

RNA polymerase uses NTPs as the substrate whereas reverse transcriptase uses dNTPs. Applicant tested the RNA polymerase activity (IVT yield) and the reverse transcriptase activity (FSS yield) under different NTP: dNTP ratios, and found that FSS is less sensitive to the presence of NTPs than IVT to dNTPs. Applicant found that NTP: dNTP ratios between 5:1 and 1:1 provided the best activity levels for both enzymes (FIG. 4A).

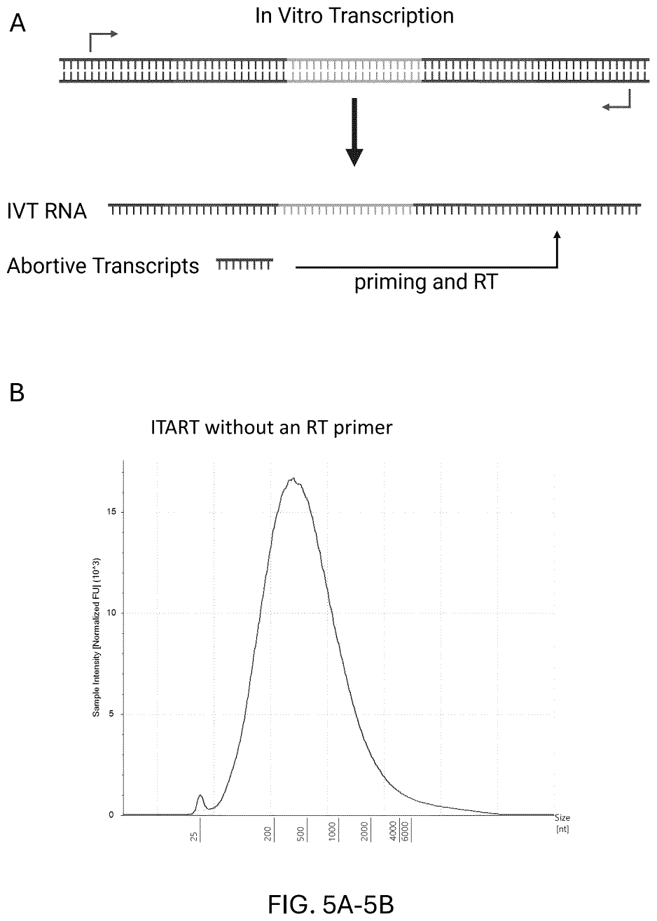

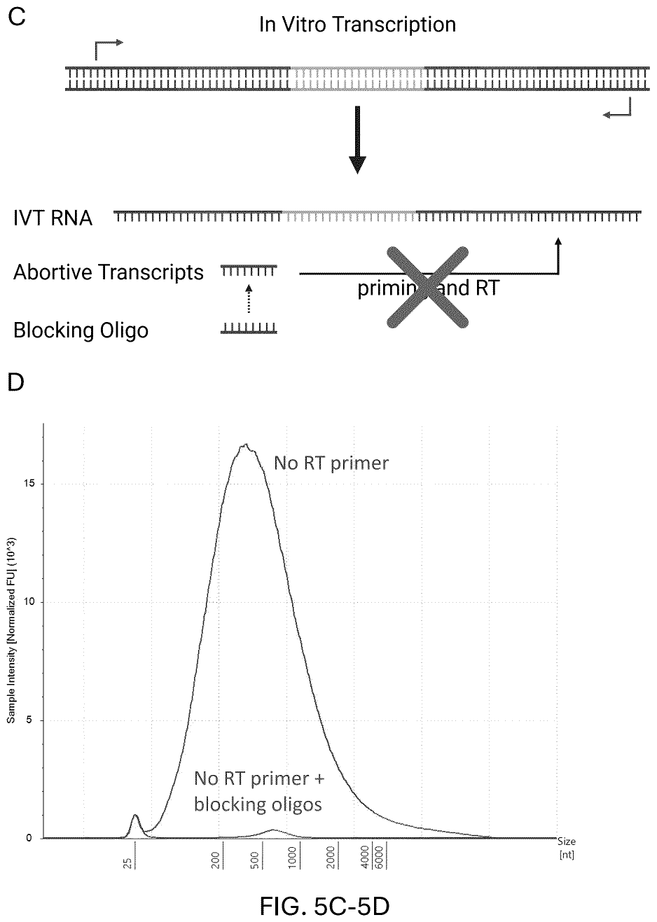

As the ITART reaction includes both IVT and RT reactions, Applicant found that abortive RNA transcripts from the IVT reaction (i.e., RNA transcript fragments that are shorter than full length resulting from early/premature termination of transcription) can serve as primers for the RT reaction from off-target sites in the absence of RT primers (FIGS. 5A-5B). Applicant designed blocking oligos that would bind abortive transcripts, and observed that such blocking oligos reduced RT from abortive transcripts (FIGS. 5C-5D).

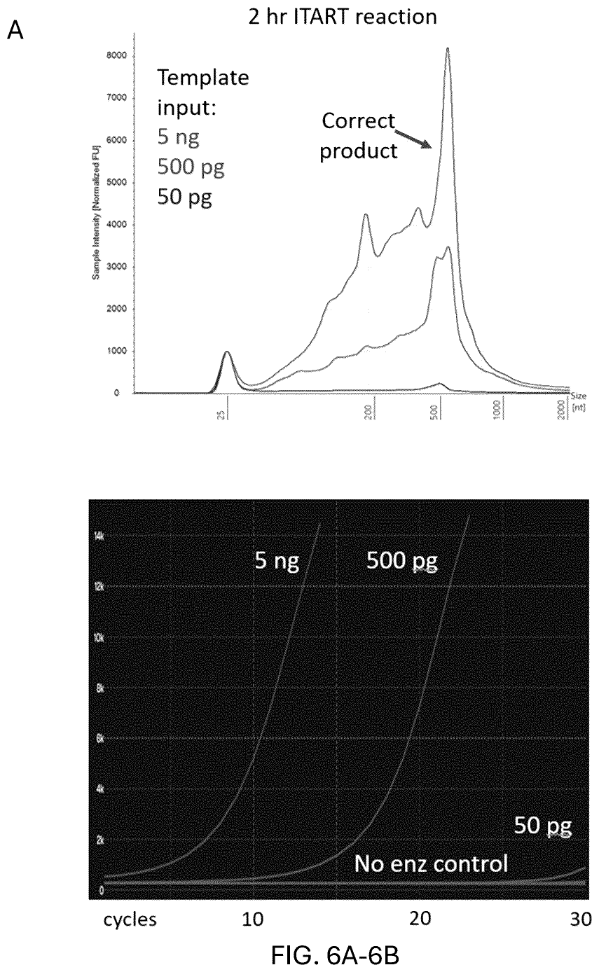

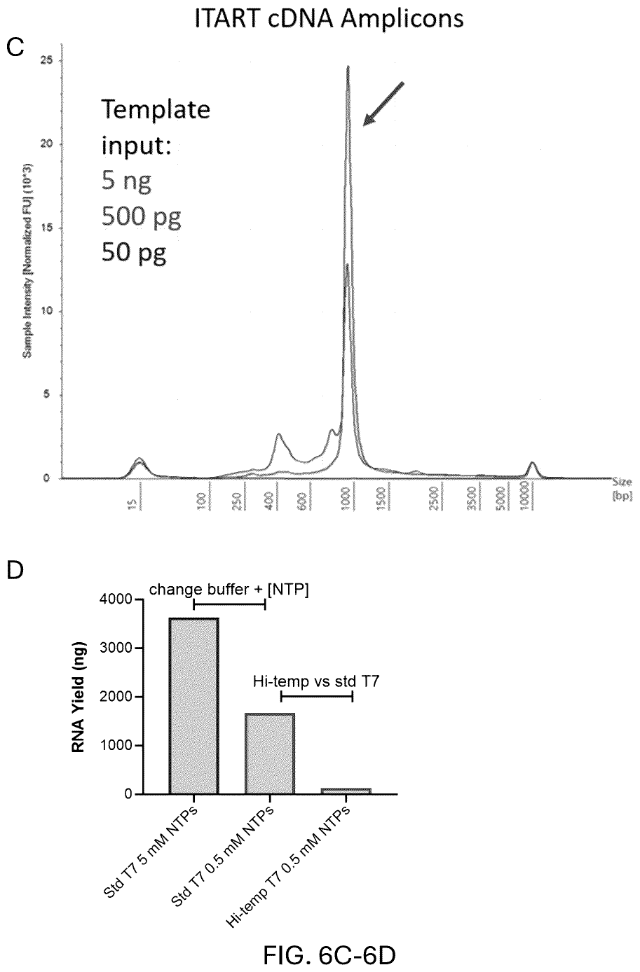

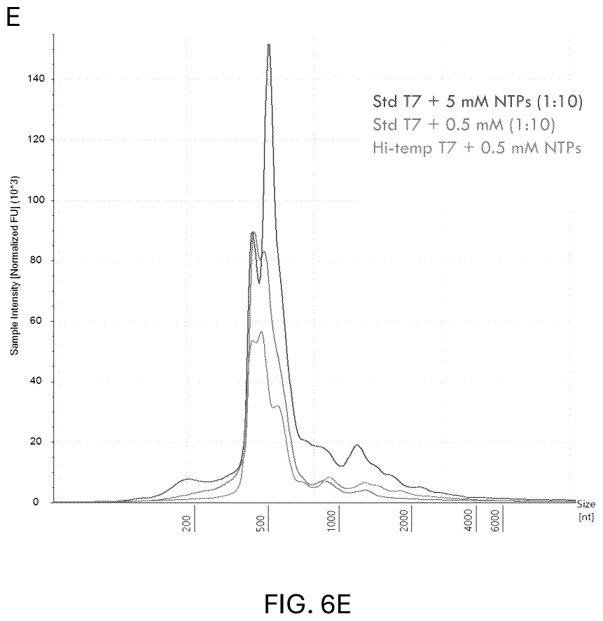

When the template is low, PCR techniques need an increased number of cycles to produce a detectable amplified product. However, the increased number of cycles also increases the possibility of false positives and accumulated amplification errors. In contrast, the ITART method produced correct size amplification products from as low as 500 pg of template (FIGS. 6A and 6C) and with less than 20 cycles of following qPCR on samples diluted 1:50 (FIG. 6B).

In another experiment, Applicant compared standard and Hi-temp T7 enzyme at different NTP concentrations for their effect on IVT yield. Standard T7 enzyme was 10 times more efficient than Hi-temp T7 in these experiments. Applicant also found that lower NTP concentrations led to a decrease in IVT yield (FIGS. 6D-6E).

### Example 3: Application of ITART for Targeted Feature Capture in Partitioned Single-Cell Workflows

This example describes the application of ITART for targeted feature capture in partition-based single-cell sequencing workflows, with a focus on guide RNA capture for Perturb-seq applications.

Perturb-seq and related single-cell CRISPR screening workflows combine single-cell RNA sequencing with CRISPR perturbations to link genotype to phenotype at single-cell resolution. In these workflows, cells are exposed to a library of guide RNAs, with different cells receiving different perturbations. To interpret the phenotype of each cell, the workflow determines which guide RNA was present in that cell by capturing and sequencing the guide RNA sequence.

Current guide RNA capture approaches suffer from several limitations, including inefficient capture, background noise from cross-cell contamination, and PCR amplification bias. These issues reduce the reliability of guide assignment and limit the performance of Perturb-seq and similar applications.

### ITART Method for Feature Capture

The ITART method was adapted to address these limitations by implementing the protocol in partition-based reactions. In this embodiment, the amplified material is derived from a single-cell sequencing library containing a genomically encoded feature with an RNA polymerase promoter upstream of the feature sequence. The feature sequence may be a guide RNA sequence, a DNA barcode sequence, an open reading frame sequence, an overexpression construct sequence, or another sequence of interest.

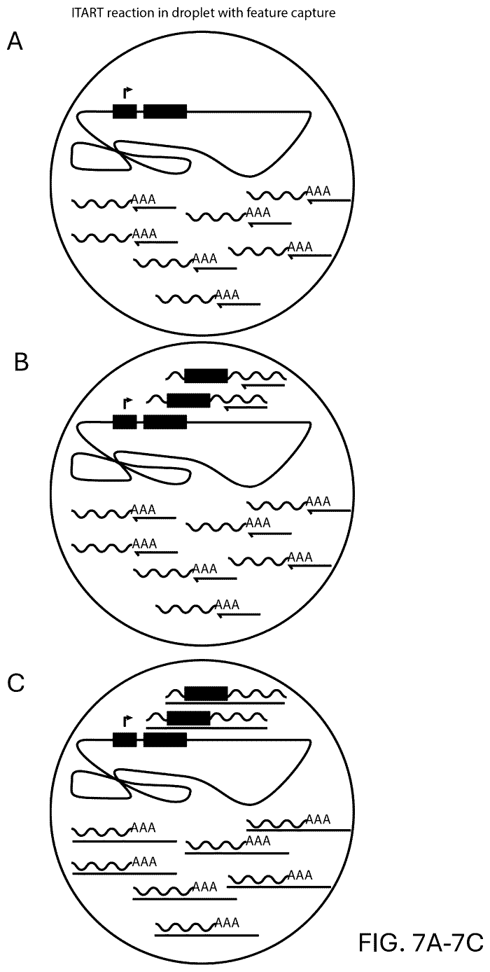

During single-cell capture, the molecular reaction includes simultaneous in vitro transcription and reverse transcription within each partition (FIGS. 7A-7C). The partition may be a droplet, emulsion, encapsulation, microwell, or other compartment that preserves the association between the feature sequence and the originating cell or partition. The reaction produces an RNA transcript from the DNA-encoded feature using an RNA polymerase, and the RNA transcript is then converted into a cDNA copy using a reverse transcriptase in the same reaction.

A T7 promoter was placed upstream of the feature sequence, and a feature sequence-specific primer was used to enrich the region of interest downstream of the T7 promoter. The workflow is compatible with cell-specific barcoding, such that each captured feature remains linked to the originating cell or partition.

### Advantages for Guide RNA Capture in Perturb-seq

The ITART-based approach provides several advantages for guide RNA capture compared to standard PCR-based methods:

First, the linear amplification reduces PCR amplification bias, improving quantitation and reducing skew in guide representation. This is particularly beneficial when working with guide libraries where accurate representation is needed.

Second, because the signal-generating transcript is created inside the partition from the DNA-encoded feature, rather than relying solely on native ambient molecules, the method reduces cross-cell contamination. The amplification occurs post-compartmentalization, minimizing background noise from lysed cells or ambient guide RNAs.

Third, the method is compatible with existing single-cell barcoding schemes, allowing integration into established workflows without requiring substantial modification of the platform.

### Application to Other Engineered Features

While guide RNA capture represents a primary application, the method is also applicable to other genomically encoded features that may not be natively transcribed. For example, DNA barcode sequences, overexpression constructs, and other engineered sequence elements can be made captureable in an RNA-based workflow by placing a T7 promoter upstream and generating an in vitro RNA transcript during the reaction.

The feature sequence may be delivered by lentivirus, stable integration systems, or other gene delivery approaches. In cases where the full transcribed product can vary in length, the assay may focus on a defined downstream portion of interest, such as a 500-base region. The primer is designed to capture that particular region, rather than the entire transcript, allowing the captured functional target region to be standardized or constrained to a narrow size distribution.

This approach is in contrast to whole-genome amplification applications, where broad fragment length variation would introduce length-dependent bias. By focusing on features with uniform or narrow size distributions, the method minimizes the impact of length-dependent capture bias that may occur in downstream processing steps.

### Experimental Implementation

The workflow was implemented as follows: Single cells were encapsulated in droplets along with barcoded beads. The cells contained genomically integrated guide RNA sequences with T7 promoters positioned upstream. Following cell lysis within the droplet, the reaction mixture contained T7 RNA polymerase, Maxima H-Minus reverse transcriptase, ribonucleotide triphosphates, deoxyribonucleotide triphosphates, and a feature-specific reverse transcription primer.

The reaction was carried out at a constant temperature of approximately 37 degrees Celsius. T7 RNA polymerase transcribed the guide RNA sequence from the genomic DNA template, producing an RNA transcript. Concurrently, the reverse transcriptase converted the RNA transcript into cDNA. The cDNA product was then captured through hybridization to the barcoded bead, linking the guide identity to the cell barcode.

The resulting library was sequenced using standard next-generation sequencing platforms. Analysis of the sequencing data demonstrated improved guide capture rates and reduced background noise compared to standard PCR-based amplification methods. The linear amplification provided more balanced representation of guide sequences, and the compartmentalized reaction format reduced cross-contamination between cells.

This example demonstrates the utility of the ITART method for targeted feature capture in partition-based single-cell workflows. The approach is particularly well-suited for applications such as Perturb-seq, where accurate guide RNA capture is needed for reliable phenotype-genotype associations. The method can also be extended to other engineered features and represents a versatile platform for feature-based single-cell library preparation.

1.-84. (canceled)

85. An isothermal method for amplifying a DNA molecule in a sample, said method comprising:
(a) producing an RNA copy of the DNA molecule using an RNA polymerase, and
(b) producing a complementary DNA (cDNA) copy of the RNA copy using a reverse transcriptase, thereby linearly amplifying the DNA molecule,
wherein (a) and (b) are carried out as a one pot reaction.

86. The isothermal method of claim 85, wherein the RNA polymerase and the reverse transcriptase are active at the same time, and (a) and (b) occur simultaneously.

87. The isothermal method of claim 85, wherein reverse transcribing the RNA copy into the cDNA copy is done while the RNA copy is being produced.

88. The isothermal method of claim 85, wherein the reverse transcriptase creates a DNA copy of RNA produced by the RNA polymerase substantially simultaneously with production of the RNA.

89. The isothermal method of claim 85, wherein the DNA molecule comprises an RNA polymerase promoter and a reverse transcriptase primer binding region.

90. The isothermal method of claim 89, wherein the RNA polymerase promoter and the reverse transcriptase primer binding region are added to the DNA molecule using a transposase.

91. The isothermal method of claim 90, wherein the transposase is selected from the group consisting of: a Tn5 transposase, a Tn3 transposase, a Tn7 transposase, a Tn10 transposase, a PiggyBac transposase, a Sleeping Beauty transposase, or a Tol2 transposase.

92. The isothermal method of claim 89, wherein the RNA polymerase promoter is introduced by an adapter, a primer, or a transposase, or wherein the RNA polymerase promoter is a unidirectional promoter or a bidirectional promoter.

93. The isothermal method of claim 85, wherein the RNA polymerase promoter is a T7 promoter.

94. The isothermal method of claim 85, wherein the DNA molecule further comprises at least one molecular barcode, wherein the molecular barcode identifies a partition or a cell of origin.

95. The isothermal method of claim 85, wherein the RNA polymerase is selected from a T7 RNA polymerase, a T3 RNA polymerase, or an SP6 RNA polymerase.

96. The isothermal method of claim 85, further comprising: contacting abortive RNA transcripts with one or more blocking oligonucleotides that reduce reverse transcription initiated from the abortive RNA transcripts.

97. The isothermal method of claim 85, further comprising: amplifying the linearly amplified DNA molecule using a polymerase chain reaction (PCR) using less than 20 cycles.

98. The isothermal method of claim 85, wherein the cDNA copy comprises a RNA polymerase promoter sequence and is used as a template for producing a second RNA copy using the RNA polymerase, and producing a second complementary DNA copy of the second RNA copy using the reverse transcriptase, wherein producing the second RNA copy and producing the second complementary DNA copy are carried out in the same one pot reaction.

99. An isothermal method for preparing a sequencing library from a sample, said method comprising:
(a) providing a nucleic acid molecule, said nucleic molecule comprising: a feature sequence and an RNA polymerase promoter upstream of the feature sequence;
(b) producing an RNA transcript of the feature sequence using an RNA polymerase; and
(c) producing a complementary DNA (cDNA) copy of the RNA transcript using a reverse transcriptase,
wherein (b) and (c) are carried out as a one pot reaction, thereby amplifying and capturing the feature sequence.

100. The method of claim 99, wherein the feature sequence is selected from a guide RNA sequence, a DNA barcode sequence, an open reading frame sequence, an overexpression construct sequence, or a sequence of interest.

101. A composition comprising: an RNA polymerase and a reverse transcriptase in the same reaction buffer, wherein the RNA polymerase and the reverse transcriptase are active in the reaction buffer at the same time.

102. The composition of claim 101, wherein the RNA polymerase is selected from a T7 RNA polymerase, a T3 RNA polymerase, or an SP6 RNA polymerase.

103. The composition of claim 101, further comprising: one or more primers configured to hybridize to a feature sequence transcribed from a DNA molecule comprising an RNA polymerase promoter upstream of the feature sequence; or one or more blocking oligonucleotides configured to hybridize to abortive RNA transcripts.
